SEQUENCE LISTING (13 sequences)

SEQ ID NO: 1 - DNA, 1839 bp, synthetic construct
  source 1..1839: mol_type = other DNA
atgcttaaacgttgtctctctccgctcaccctggttaatcaggttgcgcttattgtgttgctttctactgctattggactggcagggatggcggtttctggctggctggtgcaaggcgttcagggcagcgcccatgcgatcaacaaagcgggatcgctgcgcatgcaaagttaccgtctgttggcggcagtgccattaagcgagaaagacaagcccttaattaaagagatggaacaaacggcatttagcgccgagttgactcgagcagcagaacgagacggacaactggcgcaattacagggtttacaagattactggcgtaatgaactgatccctgcgctgatgcgtgcacaaaaccgagaaacggtgtcagcggatgtcagccagtttgttgccgggcttgatcaactggtatctggttttgaccgcaccacggaaatgcgcatcgagacagtggtactggtccatcgggtaatggcggtatttatggcacttttactggtgttcactattatctggttgcgggcgcgactgctacaaccgtggcggcaactgctggcaatggcgagtgccgtcagtcatcgcgattttacccaacgcgcaaacatcagcgggcgcaacgaaatggcgatgcttggaactgcgttgaacaatatgtctgcagaactggccgaaagttatgccgtacttgagcagcgggttcaggagaaaaccgccgggctggagcataaaaatcagatcctctcttttttatggcaggctaaccgccgtttgcattcccgcgccccgctgtgtgaacgcctgtcacctgtactcaacggcttacagaatttaaccctgctacgtgatatcgaattgcgggtgtatgacactgatgatgaagagaatcatcaggagtttacctgccagccagatatgacttgtgatgataaaggctgccagctctgcccgcgcggcgtattacccgttggtgatcgcggcacgaccctgaagtggcggctggctgactctcatacgcagtacggtattttgctggcgaccctgccacaggggcgtcatcttagccatgatcaacaacaactggtggataccctggctgaacaactcaccgccacgctggcgctggatcgccatcaggaacgtcagcaacagttgatcgtgatggaagagcgtgccaccattgcgcgcgaactgcatgattctattgcccaatctctctcttgcatgaagatgcaggtgagttgtttacagatgcagggcgatgcgctgccagaaagcagccgcgaactgttaagtcagatccgtaacgaactgaatgcatcctgggcgcagttgcgtgaattgctcatcacattccgcttgcagctcaccgagcctggattacgtccggcgctggaggcgagttgcgaagagtacagcgccaaatttggcttcccggtgaagctggattatcaattgccgcctcgcctggtgccttcgcatcaggcaatccacttgttgcaaattgcccgtgaggcattaagtaacgccctcaaacattcgcaagcgagtgaagtcgtggtgacggtggcgcaaaacgataatcaggtcaaactgaccgtccaggataacggctgcggcgtgcctgaaaatgccatccgcagcaatcactacggcatgataataatgcgcgatcgtgcgcaaagtttacgaggcgattgccgcgtccgccgtcgtgaatcaggtggcaccgaagtggtggtcacctttattcccgaaaaaactttcacagacgtccaaggagatacccatgagggaggaggaagcgagcagaaactcatctctgaagaggatctgtaa

SEQ ID NO: 2 - DNA, 696 bp, synthetic construct
  source 1..696: mol_type = other DNA
atgagtaatcaggaaccggctactatcctgctgattgacgatcacccgatgctgcgaactggcgtaaaacagcttatcagtatggcaccagatatcaccgtggttggcgaagcgagtaatggcgaacagggtattgaactggcggagtctcttgatcccgatctgatcctgttagatctcaatatgcccggcatgaacggtctggaaacgctggataaactgcgcgaaaagtccctctcagggcgcattgtggtattcagcgtctctaaccatgaagaagatgtggtcaccgcactgaaacgcggcgcggatggctatctgttaaaagatatggaaccggaagatctgctgaaagcattgcatcaggcagctgctggcgaaatggtattaagccctgatatccttaaacgtctgcaagaaatccaatttgagcggatgaaaaagcagcgcaatgagacgcagctgacagaaaaggaagtcattgttctaaaagcaattgctaaaggtcttaaaagcaaagcgattgcctttgatttgggcgtctctgagcgaacagtaaagtccagattaacgtccatttacaataaattaggcgcgaattcaagaactgaggcagtaacgattgccatgcaaaaaggtattctgacaatagacaacggaggaggaagcgattacaaggatgacgacgataagtaa

SEQ ID NO: 3 - DNA, 734 bp, synthetic construct
  source 1..734: mol_type = other DNA
actgcatatttgaaaattgcccaaacgtacatgcccgaatgtacgtttttttcatttcattgtcaactacaatgagaaagaatgtgatcaagcaatgtgttgaaaggagattatcacgtcgactctcgagtgagattgttgacggtaccgtattttggatctaggaggaaggatctatggtgtttacgctggaggatttcgtcggtgactggcgtcagacagctgggtataaccttgaccaggtacttgaacaaggcggcgtttccagcttatttcaaaatctgggggtgtctgtcacaccaattcagcgcattgtcttgtctggggagaatggtcttaaaattgatattcatgttatcatcccttacgaaggtttgtccggtgaccaaatgggacaaatcgagaaaattttcaaagtggtttatccagtggatgatcatcactttaaagtcattttacactatggtacgctggtaatcgacggtgtgacaccgaacatgattgattatttcgggcgtccgtatgaaggaatcgccgtttttgatgggaagaaaatcaccgtaacaggtactctgtggaacggaaacaaaatcatcgacgaacgcttgattaaccccgatggtagtttgttgttccgtgttacgattaacggagttacgggttggcgcctttgtgagcgtatcttggccggaagcggaagcgagcagaaactcatctctgaagaggatctgtaa

SEQ ID NO: 4 - DNA, 893 bp, synthetic construct
  source 1..893: mol_type = other DNA
actgcatatttgaaaattgcccaaacgtacatgcccgaatgtacgtttttttcatttcattgtcaactacaatgagaaagaatgtgatcaagcaatgtgttgaaaggagattatcacgtcgactctcgagtgagattgttgacggtaccgtattttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 5 - DNA, 1935 bp, synthetic construct
  source 1..1935: mol_type = other DNA
atggtgagcaaaccgtttcagcgtccgtttagcctggcaacccgtctgacctttttcatttcactggcaaccattgcagcatttttcgcatttgcatggattatgatccatagcgtgaaagtgcattttgccgaacaggatattaacgacctgaaagaaattagcgcaaccctggaacgtgttctgaatcatcctgatgaaacccaggcacgtcgtctgatgaccctggaagatattgttagcggttatagcaatgttctgattagtctggcagatagccagggtaaaaccgtttatcatagtccgggtgcaccggatattcgtgaatttacccgtgatgcaattccggataaagatgcacaaggtggtgaagtttatctgctgagcggtccgaccatgatgatgcctggtcatggtcatggccacatggaacatagcaattggcgtatgattaatctgccggttggtccgctggttgatggtaaaccgatttataccctgtatattgccctgagcattgattttcatctgcactatatcaacgatctgatgaacaaactgattatgaccgcaagcgttattagcatcctgattgtttttattgttctgctggcagttcataaaggtcatgcaccgattcgtagcgttagccgtcagattcagaatattaccagcaaagatctggatgttcgtctggatccgcagaccgttccgattgaactggaacagctggttctgagctttaatcatatgagcgcagaactggcagaaagctatgcagttctggaacagcgtgttcaagagaaaaccgcaggcctggaacataaaaatcagattctgagctttctgtggcaggcaaatcgtcgtctgcatagccgtgcaccgctgtgtgaacgtctgagtccggttctgaatggtctgcagaacctgacactgctgcgtgatattgaactgcgtgtttatgataccgatgatgaagaaaaccaccaagaatttacctgtcagccggatatgacctgtgatgataaaggttgtcagctgtgtccgcgtggtgttctgccggtgggtgatcgtggtacaaccctgaaatggcgtctggccgatagtcatacccagtatggtatcctgctggcgacactgccgcagggtcgtcatctgagccatgatcagcagcagctggtagataccctggccgaacagctgaccgcaacactggcactggatcgtcatcaagaacgtcagcagcaactgattgttatggaagaacgtgccaccattgcgcgtgaactgcatgatagcattgcacagagcctgagctgtatgaaaatgcaggttagctgtctgcagatgcagggtgatgcactgccggaaagctcacgcgaactgctgagccagattcgcaatgaactgaatgccagctgggcacagctgcgtgagctgctgattacctttcgtctgcagctgacagaaccgggtctgcgtccggcactggaagcaagctgtgaagaatattcagcgaaatttggttttccggtgaaactggattatcagctgcctccgcgtctggttccgagccatcaggcaattcatctgctgcaaattgcacgtgaagcactgagcaatgcactgaaacattcacaggcaagcgaagttgttgttaccgttgcacagaatgataaccaggttaaactgaccgttcaggataatggttgtggtgttccggaaaatgcaattcgtagcaatcattatggcatgatcattatgcgtgatcgtgcccagagcctgcgtggtgattgtcgtgttcgtcgtcgtgaaagcggtggtacagaagtggtggttacctttattcctgagaaaacctttaccgatgttcagggtgatacccatgaaggtggtggtagcgaacagaaactgatttcagaagaagatctgtaa

SEQ ID NO: 6 - DNA, 1971 bp, synthetic construct
  source 1..1971: mol_type = other DNA
atgaatacccgtcgtctgtttgcacgtagccgtctgcagctggcattttggtatgcactggttatgggtggtattctgaccctgttaggtctgggtgtttatcgtgcaattgttcaggcaaattggatggcactggaacgtgaagttgaaagcattgcaggcaccctgcatgatagcctggaaccgatgctgccgagcaatgcaagcccgaccggtgtgctgcagaaaatgctgccggatctgtgtctggttaatcagccgtgccaggttaatccgacactgattgaacgtcataccctgggtattagcgatcgtagcctgtattatatccgcctgtttgattatcagggtaatctgctggcgtttagcccgaatcagcctgcaagcctgagcagcatctttaatcaagaaacctggcagaccattcatccgcctaccggtgatcgttatcgtcagtttaccaccattctgcatagcgcaggtaataccgataaaagcagctggggttatctgcaaattggtcgtagtctggcagcctttgatgccgaaaataaacgtattctgtggattctgggtctgagctttccgattgcactgggtttagttgcatttagcagttggggtttagcaggtctggcaatgcgtccgatttatcagagctatcagcagcagcaacagtttaccgcaaatgcagcacatgaactgcgtagtccgctggcaagcctgctggcaaccgttgaagcagttctgcgtattgatagcagccatagtccggaaattaacaccatgtctgcagaactggccgaaagttatgccgtacttgagcagcgggttcaggagaaaaccgccgggctggagcataaaaatcagatcctctcttttttatggcaggctaaccgccgtttgcattcccgcgccccgctgtgtgaacgcctgtcacctgtactcaacggcttacagaatttaaccctgctacgtgatatcgaattgcgggtgtatgacactgatgatgaagagaatcatcaggagtttacctgccagccagatatgacttgtgatgataaaggctgccagctctgcccgcgcggcgtattacccgttggtgatcgcggcacgaccctgaagtggcggctggctgactctcatacgcagtacggtattttgctggcgaccctgccacaggggcgtcatcttagccatgatcaacaacaactggtggataccctggctgaacaactcaccgccacgctggcgctggatcgccatcaggaacgtcagcaacagttgatcgtgatggaagagcgtgccaccattgcgcgcgaactgcatgattctattgcccaatctctctcttgcatgaagatgcaggtgagttgtttacagatgcagggcgatgcgctgccagaaagcagccgcgaactgttaagtcagatccgtaacgaactgaatgcatcctgggcgcagttgcgtgaattgctcatcacattccgcttgcagctcaccgagcctggattacgtccggcgctggaggcgagttgcgaagagtacagcgccaaatttggcttcccggtgaagctggattatcaattgccgcctcgcctggtgccttcgcatcaggcaatccacttgttgcaaattgcccgtgaggcattaagtaacgccctcaaacattcgcaagcgagtgaagtcgtggtgacggtggcgcaaaacgataatcaggtcaaactgaccgtccaggataacggctgcggcgtgcctgaaaatgccatccgcagcaatcactacggcatgataataatgcgcgatcgtgcgcaaagtttacgaggcgattgccgcgtccgccgtcgtgaatcaggtggcaccgaagtggtggtcacctttattcccgaaaaaactttcacagacgtccaaggagatacccatgagggaggaggaagcgagcagaaactcatctctgaagaggatctgtaa

SEQ ID NO: 7 - DNA, 1593 bp, synthetic construct
  source 1..1593: mol_type = other DNA
atggtgatcaagctgaagaacaagaaaaacgactacagcaaactggaacgcaagctgtatatgtatattgttgccattgttgtggtggccattgtgtttgttctgtatattcgtagcatgatccgtggcaaattaggtgattggattctgagcattctggaaaacaaatatgatctgaatcacctggatgccatgaaactgtatcagtatagcattcgcaacaacatcgacatctttatctatgtggcgattgtgattagcattctgattctgtgtcgtgtgatgctgagtaaattcgccaaatatttcgatgaaatcaacaccggcattgatgtgctgattcagaatgaagataagcagattgaactgagcgcagaaatggatgttatggaacagaaactgaacaccatgtctgcagaactggccgaaagttatgccgtacttgagcagcgggttcaggagaaaaccgccgggctggagcataaaaatcagatcctctcttttttatggcaggctaaccgccgtttgcattcccgcgccccgctgtgtgaacgcctgtcacctgtactcaacggcttacagaatttaaccctgctacgtgatatcgaattgcgggtgtatgacactgatgatgaagagaatcatcaggagtttacctgccagccagatatgacttgtgatgataaaggctgccagctctgcccgcgcggcgtattacccgttggtgatcgcggcacgaccctgaagtggcggctggctgactctcatacgcagtacggtattttgctggcgaccctgccacaggggcgtcatcttagccatgatcaacaacaactggtggataccctggctgaacaactcaccgccacgctggcgctggatcgccatcaggaacgtcagcaacagttgatcgtgatggaagagcgtgccaccattgcgcgcgaactgcatgattctattgcccaatctctctcttgcatgaagatgcaggtgagttgtttacagatgcagggcgatgcgctgccagaaagcagccgcgaactgttaagtcagatccgtaacgaactgaatgcatcctgggcgcagttgcgtgaattgctcatcacattccgcttgcagctcaccgagcctggattacgtccggcgctggaggcgagttgcgaagagtacagcgccaaatttggcttcccggtgaagctggattatcaattgccgcctcgcctggtgccttcgcatcaggcaatccacttgttgcaaattgcccgtgaggcattaagtaacgccctcaaacattcgcaagcgagtgaagtcgtggtgacggtggcgcaaaacgataatcaggtcaaactgaccgtccaggataacggctgcggcgtgcctgaaaatgccatccgcagcaatcactacggcatgataataatgcgcgatcgtgcgcaaagtttacgaggcgattgccgcgtccgccgtcgtgaatcaggtggcaccgaagtggtggtcacctttattcccgaaaaaactttcacagacgtccaaggagatacccatgagggaggaggaagcgagcagaaactcatctctgaagaggatctgtaa

SEQ ID NO: 8 - DNA, 1866 bp, synthetic construct
  source 1..1866: mol_type = other DNA
atgatcggcttcaaaagcttctttatgcgcaccattatctttcaggttctggcaattctgctgctgtggggtctgctggttgcatgggtgaaatattggtattatccggacatggaaaaatactttgataaccagcagcgtattgttgcagcaggtattgcaaatattctggatgaaaccggcaccgacaatattgattatcgcggtattatcaaaaccatcgagggcatgtatatcgacagcattaataacggtatgcaggatgagatcgattatcatccgctgtttgttgtgtatgatcgtgataatcgtgtgctgtatagcagtcagacccagggtgaaccgctgcgtctgcctccgagcgttctgagcggtagcgttaattatgccggtgcaaattggcatctggcaggtagctggaaagaaaaacgtcagtatcgtgttattgtgggcgaaagctttaatgatcgtaccacactgtttggtaatccggcagatgttccgctgctgggtattctggcagcaattattgttaccctgctgtttaccgcatatttcagcctgcgtccgctgcgccagattgcacgtaccattagcgatcgtcagcctggtaatctgagcccgattaatgttagcgaacagtatcaagaaattcgtccggttgttatggaagtgaacaaaatgtctgcagaactggccgaaagttatgccgtacttgagcagcgggttcaggagaaaaccgccgggctggagcataaaaatcagatcctctcttttttatggcaggctaaccgccgtttgcattcccgcgccccgctgtgtgaacgcctgtcacctgtactcaacggcttacagaatttaaccctgctacgtgatatcgaattgcgggtgtatgacactgatgatgaagagaatcatcaggagtttacctgccagccagatatgacttgtgatgataaaggctgccagctctgcccgcgcggcgtattacccgttggtgatcgcggcacgaccctgaagtggcggctggctgactctcatacgcagtacggtattttgctggcgaccctgccacaggggcgtcatcttagccatgatcaacaacaactggtggataccctggctgaacaactcaccgccacgctggcgctggatcgccatcaggaacgtcagcaacagttgatcgtgatggaagagcgtgccaccattgcgcgcgaactgcatgattctattgcccaatctctctcttgcatgaagatgcaggtgagttgtttacagatgcagggcgatgcgctgccagaaagcagccgcgaactgttaagtcagatccgtaacgaactgaatgcatcctgggcgcagttgcgtgaattgctcatcacattccgcttgcagctcaccgagcctggattacgtccggcgctggaggcgagttgcgaagagtacagcgccaaatttggcttcccggtgaagctggattatcaattgccgcctcgcctggtgccttcgcatcaggcaatccacttgttgcaaattgcccgtgaggcattaagtaacgccctcaaacattcgcaagcgagtgaagtcgtggtgacggtggcgcaaaacgataatcaggtcaaactgaccgtccaggataacggctgcggcgtgcctgaaaatgccatccgcagcaatcactacggcatgataataatgcgcgatcgtgcgcaaagtttacgaggcgattgccgcgtccgccgtcgtgaatcaggtggcaccgaagtggtggtcacctttattcccgaaaaaactttcacagacgtccaaggagatacccatgagggaggaggaagcgagcagaaactcatctctgaagaggatctgtaa

SEQ ID NO: 9 - protein, 18 aa, synthetic construct
MLGTALNNMSAELAESYA

SEQ ID NO: 10 - protein, 13 aa, synthetic construct
PEINTMLHTVERQ

SEQ ID NO: 11 - protein, 21 aa, synthetic construct
PVVMEVNKLMARIDAANQREK

SEQ ID NO: 12 - protein, 18 aa, synthetic construct
QLVLSFNHMIERIEDVFT

SEQ ID NO: 13 - protein, 22 aa, synthetic construct
VMEQKLNTLKRTLEKREQDAKL

(19) United States
(12) Patent Application Publication
(10) Pub. No.: US 2025/0137032 A1
(43) Pub. Date: May 1, 2025

(54) TWO-COMPONENT SYSTEMS FOR USE IN CELL-FREE GENE EXPRESSION

(71) Applicant: Northwestern University, Evanston, IL (US)

(72) Inventors: Neha Prashant Kamat, Evanston, IL (US); Justin Alexander Peruzzi, Evanston, IL (US)

(21) Appl. No.: 18/932,214

(22) Filed: Oct. 30, 2024

Related U.S. Application Data
(60) Provisional application No. 63/594,287, filed on Oct. 30, 2023.

(51) Int. Cl. C12Q 1/48; C12Q 1/66; G01N 33/84

(52) U.S. Cl. CPC C12Q 1/485; C12Q 1/66; G01N 33/84; G01N 2333/9123

(57) ABSTRACT

Provided herein are cell-free systems comprising two-component systems including a histidine kinase and a response regulator. In embodiments, the systems further include synthetic membranes. Also provided are methods for using the systems to detect analytes and synthesize target molecules.

### CROSS-REFERENCE TO RELATED APPLICATIONS

This application claims the benefit of and priority to U.S. Provisional Application No. 63/594,287 filed on Oct. 30, 2023. The content of which is incorporated by reference in its entirety.

### STATEMENT REGARDING FEDERALLY SPONSORED RESEARCH

This invention was made with government support under grant numbers 2145050, 1844219, and 1844336 awarded by the National Science Foundation. The government has certain rights in the invention.

### REFERENCE TO SEQUENCE LISTING

The contents of the electronic sequence listing (70258102574.xml; Size: 23,714 bytes; and Date of Creation: Oct. 29, 2024) is herein incorporated by reference in its entirety.

### BACKGROUND

Cells detect and respond to environmental and chemical information by using a combination of membrane proteins and genetic polymers. Recapitulation of this behavior in synthetic systems holds promise for engineering biosensors and therapeutics. To date, cell-free systems used with synthetic membranes have largely been designed to detect membrane permeable molecules or physical cues, such as light, prior to engaging gene expression systems. While this approach has greatly expanded the capabilities of cell-mimetic systems, it is limited by the narrow number of analytes and signals that can be sensed, ultimately restricting the application of cell-mimetic technologies. Therefore, improved cell-free systems are needed.

### SUMMARY

In an aspect, provided herein is a cell-free system comprising a nucleic acid encoding a histidine kinase of a two-component system; a nucleic acid encoding a response regulator of the two-component system; and at least one nucleic acid encoding a target molecule operably linked to a response regulator-binding promoter. The histidine kinase may comprise a sensing domain, a HAMP domain, and a histidine kinase domain. The nucleic acid encoding the histidine kinase may be a NarX-encoding nucleic acid, and the nucleic acid encoding the response regulator may be a NarL-encoding nucleic acid. The NarX-encoding nucleic acid; the NarL-encoding nucleic acid, and the at least one nucleic acid encoding the target molecule may be plasmids. The NarX-encoding plasmid and the NarL-encoding plasmid may be at a ratio of between 1:3 and 10:1. In embodiments, the NarX-encoding plasmid and the NarL-encoding plasmid are at a ratio of 10:1. The NarX-encoding nucleic acid comprises a sequence having at least 90% identity to SEQ ID NO: 1, SEQ ID NO: 6, or SEQ ID NO: 8.

The cell-free system may further comprise a synthetic membrane. The synthetic membrane may comprise at least one of DMPC, POPC, DOPC, POPE, POPG, and E. coli polar lipid extract. In embodiments, the synthetic membrane comprises 90% POPC and 10% of one of POPE and POPG. In embodiments, the synthetic membrane comprises 1 POPC: 2 cholesterol and a rhodamine conjugate lipid. The synthetic membrane may be a giant unilamellar vesicle (GUV).

In another aspect, provided herein is a method of detecting an analyte in a sample, the method comprising: adding to the sample: a synthetic membrane and a cell-free system, the cell-free system comprising: a nucleic acid encoding a histidine kinase of a two-component system, wherein the histidine kinase binds the analyte; a nucleic acid encoding a response regulator for the two-component system; and at least one nucleic acid encoding a reporter molecule operably linked to a response regulator-binding promoter; and detecting the reporter molecule. The histidine kinase may comprise a sensing domain, a HAMP domain, and a histidine kinase domain. The nucleic acid encoding the histidine kinase is a NarX-encoding nucleic acid and the nucleic acid encoding the response regulator is a NarL-encoding nucleic acid. In embodiments, the analyte is nitrate and the NarX-encoding nucleic acid comprises a sequence having at least 90% identity to SEQ ID NO: 1. In embodiments, the analyte is nickel and the NarX-encoding nucleic acid comprises a sequence having at least 90% identity to SEQ ID NO: 6. In embodiments, the analyte is iron and the NarX-encoding nucleic acid comprises a sequence having at least 90% identity to SEQ ID NO: 8. The reporter molecule may comprise luciferase or EGFP.

The NarX-encoding nucleic acid; the NarL-encoding nucleic acid, and the nucleic acid encoding the reporter molecule may be plasmids. The NarX-encoding plasmid and the NarL-encoding plasmid may be provided at a ratio of between 1:3 and 10:1. In embodiments, the NarX-encoding plasmid and the NarL-encoding plasmid are provided at a ratio of 10:1.

The synthetic membrane may comprise at least one of DMPC, POPC, DOPC, POPE, POPG, and E. coli polar lipid extract. In embodiments, the synthetic membrane comprises 90% POPC and 10% of one of POPE and POPG. In embodiments, the synthetic membrane the synthetic membrane comprises 1 POPC: 2 cholesterol; and the synthetic membrane further comprises a rhodamine conjugate lipid. The synthetic membrane may be a giant unilamellar vesicle (GUV).

The method may further comprise incubating the sample, the synthetic membrane, and the cell-free system for at least about 6 hours before detecting the reporter molecule. The sample may be incubated at about 30° C.

In another aspect, provided herein is a method of synthesizing a target molecule in a sample, the method comprising: adding to the sample: a synthetic membrane, and a cell-free system, the cell-free system comprising: a nucleic acid encoding a histidine kinase of a two-component system; a nucleic acid encoding a response regulator of the two-component system; and at least one nucleic acid encoding a target molecule operably linked to a response regulator-binding promoter; and adding an activator to the sample, wherein the histidine kinase binds the activator. The histidine kinase may comprise a sensing domain, a HAMP domain, and a histidine kinase domain. The nucleic acid encoding the histidine kinase may be a NarX-encoding nucleic acid and the nucleic acid encoding the response regulator may be a NarL-encoding nucleic acid.

In embodiments, the activator is nitrate and the NarX-encoding nucleic acid comprises a sequence having at least 90% identity to SEQ ID NO: 1. The nitrate may be added at a concentration of between about 10 UM and about 10 mM. In embodiments, the activator is nickel and the NarX-encoding nucleic acid comprises a sequence having at least 90% identity to SEQ ID NO: 6. In embodiments, the activator is iron and the NarX-encoding nucleic acid comprises a sequence having at least 90% identity to SEQ ID NO: 8.

The NarX-encoding nucleic acid; the NarL-encoding nucleic acid, and the nucleic acid encoding the reporter molecule may be plasmids. The NarX-encoding plasmid and the NarL-encoding plasmid may be provided at a ratio of between 1:3 and 10:1. In embodiments, the NarX-encoding plasmid and the NarL-encoding plasmid are provided at a ratio of 1:1.

The synthetic membrane may comprise at least one of DMPC, POPC, DOPC, POPE, POPG, and E. coli polar lipid extract. In embodiments, the synthetic membrane comprises 90% POPC and 10% of one of POPE and POPG. In embodiments, the synthetic membrane comprises 1 POPC: 2 cholesterol and a rhodamine conjugate lipid. The synthetic membrane may be a giant unilamellar liposome vesicle (GUV).

The method may further comprise incubating the sample, the synthetic membrane, and the cell-free system for at least about 6 hours before detecting the reporter molecule. The sample may be incubated at about 30° C.

In another aspect, provided herein is a kit comprising any of the systems described herein, wherein the synthetic membrane is separate from the NarX-encoding nucleic acid, the NarL-encoding nucleic acid, and the at least one nucleic acid encoding the target molecules.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1E. Model two component system NarX-L is readily integrated and active in synthetic membranes. (A) Cell-free reactions were assembled by combining proteins required for gene transcription and translation, plasmids encoding T7 RNAP, NarX, NarL, a reporter gene (nanoluciferase), a membrane mimetic (DMPC liposomes), and nitrate. Upon expression and binding to nitrate, NarX can phosphorylate NarL. Phosphorylated NarL then dimerizes, binds to the promoter, and initiates transcription of the reporter gene, nanoluciferase. (B) The highest luminescence is achieved when all components of the sensor are present. Components were systematically removed to characterize downstream luciferase expression. (C) Cell-free expressed NarX-L has higher nitrate induced expression of luciferase in the presence of a membrane mimetic, DMPC. (D) Sensor activity as reported by luminescence and (E) fold change of NarX-L can be tuned by altering the DNA ratio of NarX and NarL. By increasing the DNA ratio of NarX:NarL, the overall luminescence signal is decreased, but the fold change in luminesce in response to nitrate is increased. The sum of NarX and NarL plasmid concentrations were kept at 6.6 nM. All error bars represent the S. E. M. for n=3 independent replicates.

FIGS. 2A-2F. Vesicles of varying composition alter NarX and NarL expression and subsequent activity. (A) By modulating the composition of vesicles that are doped into cell-free reactions, we can characterize how membrane physical features affect NarX cotranslational insertion and activity. Vesicles were composed of 100% DMPC, 100% POPC, 100% DOPC, 30% POPE/70% POPC, 30% POPG/70% POPC, and 100% E. coli polar lipid extract. (B) NarX-L activity in membranes of different composition in the absence and presence of 1 mM nitrate. Fold change in luminescence is indicated over each membrane composition. (C) NarX expression varies with membrane composition as determined by western blot band intensity. (D) Western blot band intensity is correlated with luminescence and performance of the sensor. (E, F) The viscosity of each membrane measured via DPH anisotropy correlates with protein expression as determined by western blot. All error bars represent the S. E. M. for n=3 independent replicates. For western blots, n=6 as band intensities in the absence and presence of nitrate were grouped for each membrane condition.

FIGS. 3A-3E. Minor augmentations in membrane composition enable interrogation of lipid physical properties on NarX activity. (A) By altering 10 mol % of the lipid in vesicles composed of POPC, protein expression can be conserved and allow differences in activity to be attributed to protein-lipid interactions. (B) Western blot band intensity of NarX for all samples shows that protein expression was not substantially different when membrane composition was changed. (C) NarX activity and fold change in luminescence varied with membrane composition. (D) Spearman's correlation demonstrates that changes in luminescence and on/off ratio correlate with lipid physical properties. (E) Lipid critical packing parameter was found to linearly correlate with the fold change in luminescence. All error bars represent the S. E. M. for n=3 independent replicates.

FIGS. 4A-4H. Engineering new NarX chimeras for expanded sensing capabilities. (A) Membrane bound histidine kinases generally possess similar structural features, allowing chimeric proteins with new functions to be engineered by mixing and matching parts. (B) Sequence alignment of NarX (nitrate) (SEQ ID NO: 9), NrsS (nickel) (SEQ ID NO: 10), RssA (iron) (SEQ ID NO: 11), CusS (SEQ ID NO: 12), and VanS (vancomycin) (SEQ ID NO: 13) HAMP domains. Asterisk (*), colon (:), and periods (.) indicate fully conserved amino acids, or amino acids with strongly conserved, and weakly conserved properties respectively. The red arrow indicates the cross over point of chimeras. (C, D, E) Luminescence as a result of NrsS, RssA, and VanS chimera signaling when expressed in POPC, DOPC, or DMPC membranes with each chimera's respective ligand. Fold change in luminescence in response to each ligand is indicated above each membrane composition. (F) NarX and NrsS activate specifically to their respective ligands. (G, H) Coexpressing NarX and the NrsS chimera allows for sensing of nitrate and nickel in POPC vesicles. All error bars represent the S. E. M. for n=3 independent replicates.

FIGS. 5A-5D. Towards the deployment of two-component sensors for real world sensing. (A) The cell-free expressed NarX-L system can be encapsulated into giant unilamellar vesicles. (B) Signaling and subsequent mEGFP expression can be visualized on the microscope (top). Rhodamine labeled membranes are colored in red and mEGFP are colored in green. Scale bars are 10 μm. Expression of luciferase inside giant unilamellar vesicles can be detected in bulk solution (bottom). (C) Lyophilized reactions remain active, following rehydration with non-ideal media. (D) NarX-L enables higher expression in the presence of 100 μM nitrate compared to the absence of nitrate when rehydrated in Milli-Q water, lake water, and 50% FBS following lyophilization. Fold change in luminescence in response to nitrate is indicated above each condition. All error bars represent the S. E. M. for n=3 independent replicates.

FIGS. 6A-6C. NarX associates with synthetic membranes. (A) NarX was expressed with and without DMPC vesicles labeled with biotin. (B) Vesicles were then pulled down with avidin coated magnetic beads and subsequently washed to remove non-vesicle associated protein. A western blot was then performed on the purified samples, probing for myc-tagged NarX. (C) NarX associates with DMPC vesicles as determined by Western Blot. NarX was expressed as determined by probing samples prior to purification. However, NarX was only retained only in samples with vesicles present in the reaction following purification.

FIG. 7. Protein expression is inhibited at high nitrate concentrations. Cell-free reactions were performed with increasing amounts of nitrate and expression of NarX was probed via western blot. At high concentrations of nitrate band intensity decreased, suggesting that nitrate inhibits protein expression. Each lane pair represents a replicate (n=2).

FIG. 8. Protein expression can be tuned by altering DNA concentrations in cell-free reactions. Cell-free reactions containing 1 NarX: 1 NarL and 10 NarX: 1 NarL mol DNA (6.6 nM total) were run and protein expression was quantified by western blot. The ratio of band intensities of NarX to NarL increases with increasing ratio of the respective DNA concentrations.

FIG. 9. Nitrate titration for cell-free reactions with DMPC vesicles and either 1:1 or 10:1 NarX:NarL plasmid ratio. By altering the DNA ratio of NarX and NarL, the fold increase in luminesce in response to nitrate is increased. The sum of NarX and NarL plasmid concentrations were kept at 6.6 nM. All error bars represent the S. E. M. for n=3 independent replicates.

FIG. 10. NarL band intensity correlates with NarX band intensity. Protein expression of each component is interrelated, as unfolded or aggregated protein likely aggregates nascent and expressed proteins. Each line represents a different western blot.

FIGS. 11A-11D. Measurement of membrane defects and viscosity for membranes used in this study. (A) Laurdan GP was used to measure membrane defects, while DPH anisotropy was used to measure membrane viscosity. (B) Laurdan GP measurements of membranes in FIG. 2. (C) DPH Anisotropy and (D) Laurdan GP for membranes in FIG. 3. Differences in DPH Anisotropy were not observed as POPC made up of 90 mol % in each sample, while only 10 mol % was altered. All error bars represent the S. E. M. for n=3 independent replicates.

FIG. 12. Pearson and Spearman correlations for data in FIG. 2.

FIG. 13. Pearson and Spearman correlations for data in FIG. 3.

FIG. 14. Copper inhibits cell-free protein synthesis at relevant concentrations. Protein expression as determined by NarX Western Blot band intensity and luminescence decreased with increasing copper concentration.

FIG. 15. Tuning the concentration of reporter plasmid enhances chimera sensor fold change. Cell-free reactions were assembled with POPC liposomes and the NrsS chimeric protein, varying the concentration of the reporter plasmid from 3.3 to 0.33 nM. Fold change of luciferase expression in the presence and absence of nickel was calculated. As the concentration of reporter plasmid was decreased, the fold change in response to nickel increased (n=1).

FIG. 16. Encapsulated TCS signaling is inhibited upon incubation of Proteinase K but not RNAse. External Proteinase K likely digests externally displayed nitrate binding domains, inhibiting downstream, nitrate dependent signaling. n=2, error bars represent S. E. M.

FIG. 17A-17C. Characterizing the performance of lyophilized NarX-L sensors. (A) Background subtracted eGFP fluorescence following lyophilization and rehydration with different media. (B) Lyophilized NarX-L activity when vesicles are lyophilized with cell-free components. (C) Vesicles appear to float when lyophilized with cell-free reactions, likely leading to poor fold increase in response to nitrate. Experiments were repeated with vesicles in reactions prior to lyophilization multiple times and always produced fold increases opposite to what was observed in non-lyophilized experiments; representative data is shown (n=1).

FIG. 18. Kinetics of the NarX sensor. Cell-free reactions were set up and luminescence was read on the plate reader over time. The nitrate sample was significantly higher than the water sample after 6.5 hours (p<0.03). Luminescence decreases over time likely due to the reduction in substrate as it is used. Error bars represent the standard deviation, n=2.

### DETAILED DESCRIPTION

The present disclosure provides cell-free systems using two-component systems and methods of their use.

### Systems

In a first aspect, provided herein is a cell-free system comprising a nucleic acid encoding a histidine kinase of a two-component system; a nucleic acid encoding a response regulator of the two-component system; and at least one nucleic acid encoding a target molecule operably linked to a response regulator-binding promoter. The histidine kinase may comprise a sensing domain that detects an analyte, a HAMP domain, and a histidine kinase domain that phosphorylates the response regulator.

A “two-component system” or “two-component regulatory system” refers to a system that serves as a basic stimulus-response coupling mechanism that allows organisms to sense and respond to changes in environmental conditions. Two-component systems typically consist of a membrane-bound histidine kinase that senses a specific environmental stimulus, and a corresponding response regulator that mediates the cellular response, mostly through differential expression of target genes.

A “histidine kinase” is a multifunctional, transmembrane protein of the transferase class of enzymes that play a role in signal transduction across the cellular membrane. Histidine kinase transfers a phosphate group from ATP to a histidine residue within the kinase, and then to an aspartate residue on a receiver domain of a response regulator protein.

The sensing domain of the histidine kinase is an extracellular domain that binds to an analyte. The HAMP domain transmits conformational changes from the extracellular domain upon analyte binding to intracellular signaling kinase domains. The kinase domain activates phosphorylation of an intracellular soluble protein.

A “response regulator” is a protein that mediates a cell's response to changes in its environment as part of a two-component regulatory system. Response regulators are coupled to specific histidine kinases which serve as the sensors of environmental changes, as described above.

The Examples demonstrate that the NarX-L system can be used to activate target gene expression in cell-free protein synthesis systems. Therefore, in exemplary embodiments, the nucleic acid encoding the histidine kinase is a NarX-encoding nucleic acid and the nucleic acid encoding the response regulator is a NarL-encoding nucleic acid.

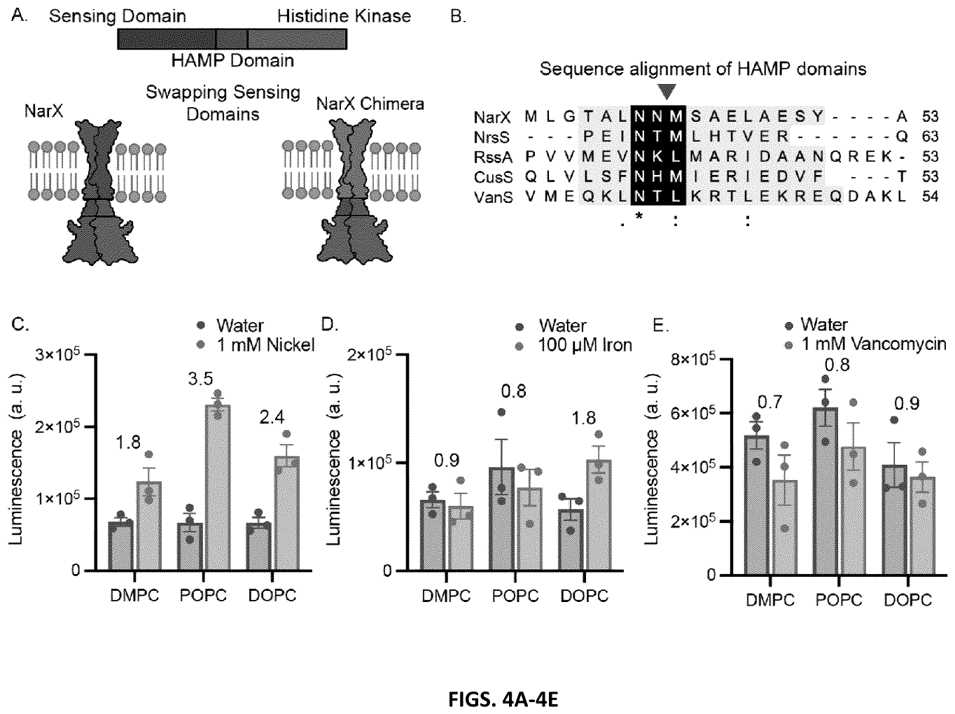

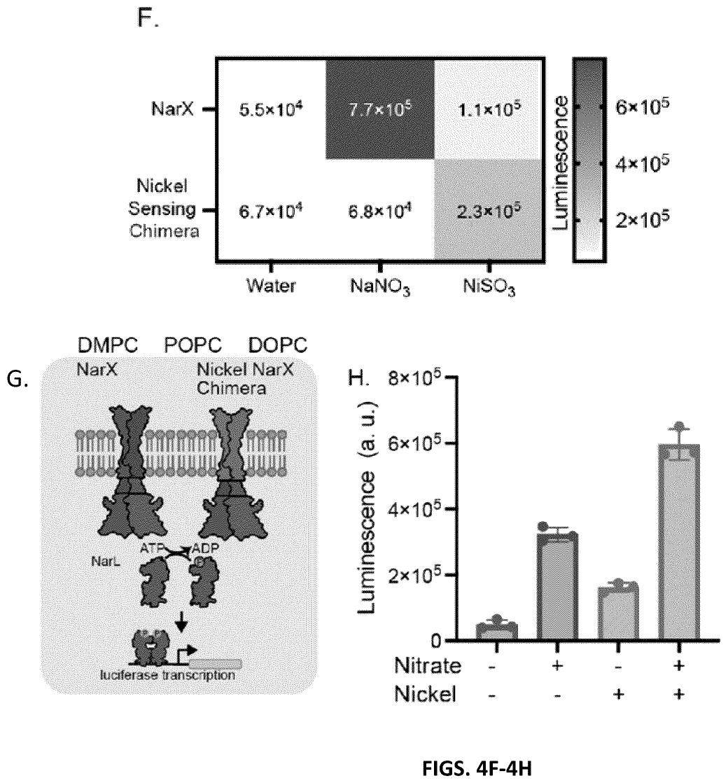

NarX-L is a two-component regulatory system expressed in E. coli. NarX is a nitrate/nitrite sensor and histidine kinase having an intracellular histidine kinase domain that activates phosphorylation of NarL in response to binding the extracellular analyte, nitrate/nitrite. Once phosphorylated, NarL binds to its cognate promoter and positively regulates gene transcription. As shown in FIGS. 4A and B, the inventors engineered NarX chimeras by replacing the native NarX sensing domain and HAMP domain with the domains of other proteins (NrsS, RssA, VanS, CusS). The NrsS and RssA chimeras were demonstrated to effectively activate NarL-mediated transcription in response to nickel and iron, respectively. Therefore, the NarX may comprise wild type NarX (SEQ ID NO: 1 or a sequence having at least 80%, 85%, 90%, 95% or 98% identity to SEQ ID NO: 1), or a NarX chimera that senses other minerals or metals. In exemplary embodiments, the chimera is a NrsS chimera that binds nickel (SEQ ID NO: 6 or a sequence having at least 80%, 85%, 90%, 95% or 98% identity to SEQ ID NO: 6) or a RssA chimera that binds iron (SEQ ID NO: 8 or a sequence having at least 80%, 85%, 90%, 95% or 98% identity to SEQ ID NO: 8).

Regarding nucleic acid sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

The cell-free system may further comprise cell extract, reaction buffer, DNA templates, and small molecules required for transcription and/or translation, e.g. NTPs, amino acids, buffering salts, crowding agents, and an energy source.

The terms “nucleic acid,” “nucleic acid sequence,” “polynucleotide,” and “polynucleotide sequence,” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell. The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The nucleic acid sequences disclosed herein may be present in vectors. For example, the vectors may comprise a polynucleotide encoding an ORF of a protein. The polynucleotide present in the vector may be operably linked to a prokaryotic or eukaryotic promoter. The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” or “recombinant expression vectors.” One type of vector is a “plasmid”, which refers to a circular double stranded DNA loop into which additional DNA segments may be ligated, specifically exogenous DNA segments encoding the fusion protein. Vectors as disclosed herein may include “plasmids” or “plasmid vectors.” “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a prokaryotic or eukaryotic promoter) operably linked to a polynucleotide that encodes a protein. A “heterologous promoter” refers to a promoter that is not the native or endogenous promoter for the protein or RNA that is being expressed.

The nucleic acids encoding NarX, NarL, and the target molecules may be plasmids. The Nar-X encoding plasmid and the NarL-encoding plasmid may be at a ratio of between 1:3 and 10:1. For example, the NarX plasmid: NarL plasmid ratio may be 1:3, 2:3, 1:1, 2:1, 3:1, 4:1, 5:1, 6:1, 7:1, 8:1, 9:1, 10:1, and any ratios and ranges in between. In preferred embodiments, the NarX plasmid and NarL plasmid are provided at a ratio of 10:1.

Cell-free protein synthesis (CFPS) is known and has been described in the art. (See, e.g., U.S. Pat. Nos. 6,548,276; 7,186,525; 8,734,856; 7,235,382; 7,273,615; 7,008,651; 6,994,986; 7,312,049; 7,776,535; 7,817,794; 8,298,759; 8,715,958; 9,005,920; U.S. Publication No. 2014/0349353, U.S. Publication No. 2016/0060301, U.S. Publication No. 2018/0016612, and U.S. Publication No. 2018/0016614, the contents of which are incorporated herein by reference in their entireties). A “cell-free system” or “CFPS reaction mixture” typically contains a crude or partially-purified cell extract, an RNA translation template, and a suitable reaction buffer for promoting cell-free protein synthesis from the RNA translation template. In other embodiments, the cell-free system can include a DNA expression template encoding an open reading frame operably linked to a promoter element for a DNA-dependent RNA polymerase. In these other aspects, the cell-free system can also include a DNA-dependent RNA polymerase to direct transcription of an RNA translation template encoding the open reading frame. In these other aspects, additional NTP's and divalent cation cofactor can be included in the cell-free system. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The disclosed cell-free systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

The cell-free system disclosed herein may comprise a cellular extract from a host strain. Because CFPS exploits an ensemble of catalytic proteins prepared from the crude lysate of cells, the cell extract (whose composition is sensitive to growth media, lysis method, and processing conditions) is an important component of extract-based CFPS reactions. A variety of methods exist for preparing an extract competent for cell-free protein synthesis, including those disclosed in U.S. Patent Application Publication No. 2014/0295492 and U.S. Patent Application Publication No. 2016/0060301, the contents of which are incorporated by reference in their entireties. The cellular extract of the platform may be prepared from a cell culture of a prokaryote (e.g., E. coli). While E. coli is exemplified herein, the bacterial species is not intended to be limiting. Other bacterial species suitable for the compositions and methods disclosed herein include but are not limited to (e.g., Bacillus species such as Bacillus subtilis, Vibrio species such as Vibrio natrigens, Pseudomonas species, etc.). A eukaryotic cell culture (e.g. Chinese Hamster Ovary cells) may be used. In some embodiments, the cell culture is in stationary phase. In some embodiments, stationary phase may be defined as the cell culture having an OD600 of greater than about 3.0, 3.5, 4.0, 4.5, 5.0, 5.5, 6.0, 6.5, 7.0, 7.5, 8.0, or having an OD600 within a range bounded by any of these values. Further methods for preparing a cell-free system are disclosed in International Patent Application Publication No. WO2020185451A2, the contents of which are incorporated by reference in its entirety.

The cell extract may be prepared by lysing the cells of the cell culture and isolating a fraction from the lysed cells. For example, the cell extract may be prepared by lysing the cells of the cell culture and subjecting the lysed cells to centrifugal force, and isolating a fraction after centrifugation.

The term “target molecule” as used herein refers to any RNA transcript or protein desired for expression within the system. A target protein may include a detectable protein, e.g. luciferase or GFP; a protein needed for increased expression within an organism; a protein desired for increased expression and extraction or purification from the system. As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. A target RNA may include a detectable RNA molecule, e.g. mRNA or other RNA transcript. Transcripts and encoded polypeptides may be collectively referred to as “gene product.” If the polynucleotide is derived from genomic DNA, expression may include splicing of the mRNA in a eukaryotic cell.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

The cell-free system may further comprise a synthetic membrane. As used herein, the term “synthetic membrane” refers to a synthetically created lipid layer or lipid structure in which the NarX protein can be embedded. The terms “lipid layer,” “lipid structure,” and “lipid membrane” mean a continuous, self-assembled barrier comprising a plurality of amphiphilic lipids. In some embodiments, the lipid layer comprises a single layer of amphiphilic lipids, e.g., a micelle or a reverse micelle, having a hydrophilic surface and a hydrophobic surface. In other embodiments, the lipid layer is a lipid bilayer comprising two layers of amphiphilic lipids having an inner-hydrophilic surface, an outer-hydrophilic surface, and a hydrophobic core disposed between the inner-hydrophilic surface and the outer-hydrophilic surface, e.g., a liposome, a lipid nanoparticle, a cell, a cellular organelle, or a 2-dimensional membrane.

“Amphiphilic lipid” means any chemical compound having both hydrophilic and hydrophobic properties and typically composed of a polar head group and lipophilic tail. The polar head group may charged or uncharged. Suitably, the polar head groups may comprise anionic head groups (such as carboxylates, sulfates, sulfonates, or phosphates), cationic head groups (such as ammoniums), or uncharged head groups (such as alcohols). The lipophilic tail is typically a saturated or unsaturated alkyl or a saturated or unsaturated alkylene having at least four carbon atoms, suitably between 6 and 24 carbon atoms. Exemplary amphiphilic lipids include, without limitation, phospholipids (e.g., sphingomyelins or phosphoglycerides such as phosphatidylserines, phosphatidylethanolamines, phosphatidylinositols, or phosphatidylcholines), glycolipids, fatty acids, amphiphilic di-block copolymers, amphiphilic tri-block copolymers, amphiphilic dendrimers, amphiphilic dendrons, or peptide amphiphiles.

The lipid layers may comprise additional components. Suitably, the lipid layer may comprise a protein, a carbohydrate, a sterol, or any combination thereof. Proteins may be surface proteins, integral proteins, transmembrane proteins, globular proteins, glycoproteins, and, as used herein, also include oligopeptides.

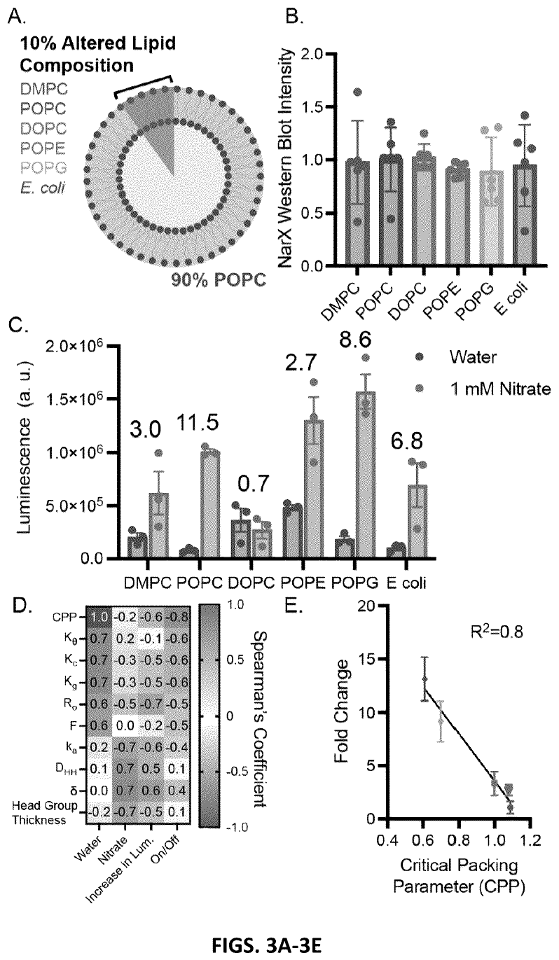

The synthetic membrane may comprise 1,2-dimyristoyl-sn-glycero-3-phosphocholine (DMPC), 1-palmitoyl-2-oleoyl-glycero-3-phosphocholine (POPC), 1,2-dioleoyl-sn-glycero-3-phosphocholine (DOPC), 1-palmitoyl-2-oleoyl-sn-glycero-3-phosphoethanolamine (POPE), 1-palmitoyl-2-oleoyl-sn-glycero-3-phospho-(1′-rac-glycerol) (sodium salt) (POPG), and E. coli polar lipid extract. E. coli polar lipid extract is a chloroform:methanol extract of E. coli, precipitated with acetone and extracted with diethyl ether. The synthetic membrane may comprise 100% DMPC, 100% POPC, 100% DOPC or 100% E. coli polar lipid extract. The lipids may have dyes conjugated to them for membrane imaging. As shown in the examples, the membrane composition impacts NarX-L activity in the cell-free systems. See, FIG. 3. In exemplary embodiments, the synthetic membrane may comprise 90% POPC and 10% of one of POPE and POPG. The synthetic membrane may comprise 1 POPC: 2 cholesterol and a rhodamine conjugate lipid.

The lipid layers disclosed herein may form vesicles. A “vesicle” means any closed-structure comprising a lipid layer enclosing a liquid or gas. Vesicles may vary in size from about 10 nm to about 100 μm in diameter. In some cases, the vesicles may be characterized as “small” (typically less than 100 nm in diameter), “large” (typically 100 nm to 1 μm), or “giant” (typically greater than 1 μm). Vesicles may be unilamellar or multilamellar. Exemplary vesicles include, without limitation, micelles, reverse micelles, small unilamellar liposome vesicles (SUVs), large unilamellar liposome vesicles (LUVs), giant unilamellar liposome vesicles (GUVs), cells, organelles, vacuoles, lysosomes, transport vesicles, secretory vesicles, exosomes, microvesicles, membrane particles, apoptotic blebs, polymersomes, dendrimersomes, peptide-amphiphile vesicles, gas vesicles, and synthetically made vesicles. In exemplary embodiments, the vesicle is a giant unilamellar liposome vesicle (GUV). The GUV may comprise 1 POPC: 2 cholesterol and a rhodamine conjugate lipid.

### Methods

In a second aspect, provided herein is a method of using the two-component system as a sensor to detect an analyte in a sample. The method comprises adding the sample to a cell-free system, the cell-free system comprising: a nucleic acid encoding a histidine kinase of a two-component system, wherein the histidine kinase binds the analyte; a nucleic acid encoding a response regulator for the two-component system; and at least one nucleic acid encoding a reporter molecule operably linked to a response regulator-binding promoter; and detecting the reporter molecule. The cell-free system may include any of the cell-free systems disclosed herein. An “analyte” as used herein refers to any mineral or metal that the histidine kinase is configured to detect. In exemplary embodiments, the analyte is nitrate (detected by wildtype NarX), nickel (detected by the NrsS chimera NarX), or iron (detected by the RssA chimera NarX).

As used herein, “sample” refers to any fluid substance that may be added to the cell-free system. Appropriate samples include without limitation, food samples, drinking water, environmental samples, and agricultural products. In embodiments, the systems and methods provided herein are used in food safety and food biosecurity applications, such as screening food products and materials used in food processing or packaging for the presence of contaminants in biological and/or non-biological samples. In embodiments, the sample is a biological sample obtained from an individual (e.g., a human subject, a non-human mammal). The sample is, in some cases, a diagnostic sample. The sample type will vary depending on the target analyte. For example, diagnostic samples can be a serum sample, blood sample, sputum sample, urine sample, or other biological fluid. In some cases, serum samples have been frozen (e.g., at −80° C.) prior to testing. In some cases, samples appropriate for use according to the methods provided herein are “non-biological” in whole or in part. Non-biological samples include, without limitation, plastic and packaging materials, paper, clothing fibers, and metal surfaces.

Other applications for which the methods provided herein include, without limitation, profiling species in an environment (e.g., water); profiling contaminants in a human or animal microbiome; food safety applications (e.g., detecting the presence of contaminants or unsafe levels of analytes); or to detect exposure of a patient to a toxin or environmental agent that affects levels of the analyte.

The term “reporter molecule” refers to a detectable or measurable molecule, expressed from a construct, having sufficient sensitivity to reflect changes in concentration or amount of analyte present in the sample. Examples of reporter molecules include, without limitation, digoxigenin, enzymatic reporters (e.g., β-galactosidase, alkaline phosphatase, DHFR, CAT), fluorescent or chemiluminescent reporters (e.g., GFP variants, mCherry, luciferase, e.g., luciferase derived from the firefly (Photinus pyralis) or the sea pansy (Renilla reniformis) and mutants thereof), aptamers, etc. In exemplary embodiments, the reporter is luciferase or EGFP. As used herein, the term “aptamer” refers to single-stranded DNA or RNA oligonucleotides that bind specifically to molecular targets with high affinity. The aptamer may bind a molecular target/analyte with a K_{d}<1 μM, 500 nM, 250 nM, 100 nM, 50 nM, 10 nM, 1 nM, 0.5 nM or 0.1 nM. Molecular targets may include, without limitation, proteins, lipids, carbohydrates, other types of molecules, or any specified binding site thereof. Aptamer based reporter molecules may comprise an oligonucleotide having a label. A label may comprise any suitable chemical or substance having sufficient sensitivity to reflect changes in concentration or amount of analyte present in the sample when the aptamer binds its target analyte. Detection of the reporter molecule may be performed by any absorbance, colorimetric, fluorometric, etc, methods known in the art.

The method of detecting an analyte may comprise incubating the sample with the cell-free system for at least about 6 hours. The incubation period may be longer than about 6.5 hours. The incubation period may be extended to up to 16 hours. The incubation may be done at between about 25° C. and 40° C. In embodiments, the incubation may be done at about 30° C.

In a third aspect, provided herein is a method of synthesizing a target molecule in a sample, the method comprising: adding to the sample a synthetic membrane and a cell-free system, the cell-free system comprising: a nucleic acid encoding a histidine kinase of a two-component system; a nucleic acid encoding a response regulator of the two-component system; and at least one nucleic acid encoding a target molecule operably linked to a response regulator-binding promoter; then adding an activator to the sample, wherein the histidine kinase binds to the activator. The cell-free system may include any of the cell-free systems disclosed herein. As used herein, the term “activator” refers to any mineral or metal that the histidine kinase is configured to detect. In exemplary embodiments, the activator is nitrate (detected by wildtype NarX), nickel (detected by the NrsS chimera NarX), or iron (detected by the RssA chimera NarX).

The activator may be added to the sample at a concentration of between about 10 μM and about 10 mM. The activator may be added at about 10 μM, 20 μM, 30 μM, 50 μM, 75 μM, 100 μM, 250 μM, 500 μM, 1 mM, 2.5 mM, 5 mM, 10 mM, or any concentration or range in between.

The method of synthesizing the target sample may comprise incubating the sample with the cell-free system for at least about 6 hours. The incubation period may be longer than about 6.5 hours. The incubation may be done at about 30° C.

### Kits

In a fourth aspect, provided herein is a kit comprising any of the cell-free systems disclosed herein, wherein the synthetic membrane is separate from the nucleic acid encoding the histidine kinase, the nucleic acid encoding the response regulator, and the at least one nucleic acid encoding the target molecule. The synthetic membrane and nucleic acids may be separated by separate containers. The nucleic acids and synthetic membrane components may be lyophilized.

The kit may include a buffer. The buffer may be dried with the nucleic acids and synthetic membrane components, and rehydrated with water. The kit may further include a dropper for dispensing controlled volumes of liquid, such as a controlled volume disposable Pasteur pipette. The kit may further include a written insert component comprising instructions for detecting an analyte in a sample according to methods of this disclosure.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLE

### Introduction

Cell-free transcription and translation systems are powerful tools to study and engineer biology (1). Initially used to decipher the genetic code (2), cell-free systems have now been used for applications ranging from the development of therapeutics (3-5) to distributable biosensors (6-8). Recently, synthetic membranes have further expanded the capabilities of cell-free systems, most often acting as a compartment to concentrate and protect encapsulated components (9-13). Yet increasingly, membranes have been recognized for their capacity to augment cell-free systems by incorporating functional transmembrane proteins (9), enabling protein posttranslational modification (14), lipid biosynthesis (15), and membrane permeability (16, 17). A critical gap in the design of membrane-augmented cell-free systems to date, however, has been the integration of transmembrane signaling capabilities that both leverage the diverse array of membrane proteins and engage with genetic systems. To date, cell-free systems used with synthetic membranes have largely been designed to detect membrane permeable molecules or physical cues, such as light, prior to engaging gene expression systems (11, 13, 18, 19). While this approach has greatly expanded the capabilities of cell-mimetic systems, it is limited by the narrow number of analytes and signals that can be sensed, ultimately restricting the application of cell-mimetic technologies. Transmembrane receptors that can transduce a wide array of chemical and environmental signals across membranes into genetically programmed responses will greatly expand the functionality of cell-free systems, enabling biosensing and programmed biosynthesis in complex aqueous environments, such as the body.

Widespread in prokaryotes, two component systems (TCS) are simple transmembrane sensing motifs which enable the transduction of environmental stimuli into a cellular response (20). The canonical TCS is composed of a transmembrane protein sensor histidine kinase and a soluble response regulator which can regulate gene expression upon phosphorylation. These systems can sense many unique physical and chemical signals, such as light, osmotic pressure, small molecules, and ions, and can trigger cellular responses ranging from cell division to taxis (20-23). Further, TCSs have been shown to be amenable to protein engineering due to their structurally conserved and modular parts (24-27), and functional in non-native organisms (28, 29). Combined, these features make membrane bound TCSs an attractive system to integrate into cell-free systems.

Here, we investigate how a model membrane-bound TCS, the nitrate sensing NarX-L, can be integrated into synthetic membranes using a cell-free protein synthesis system. We demonstrate that NarX-L can be reconstituted into synthetic membranes and characterize how membrane physiochemical properties can be used to tune the activity of the sensor. Further, we use protein engineering to explore the modularity of membrane-bound kinases to sense other ligands, and we find that the activity of the histidine kinase depends on both the membrane composition and sensing domain. Using this system, we generate nanoparticles that can sense multiple ligands, detect contaminants in nonideal matrices, and remain functional when fully encapsulated into synthetic vesicles.

### Results

Integration of the two-component system, NarX-L, into synthetic membranes.

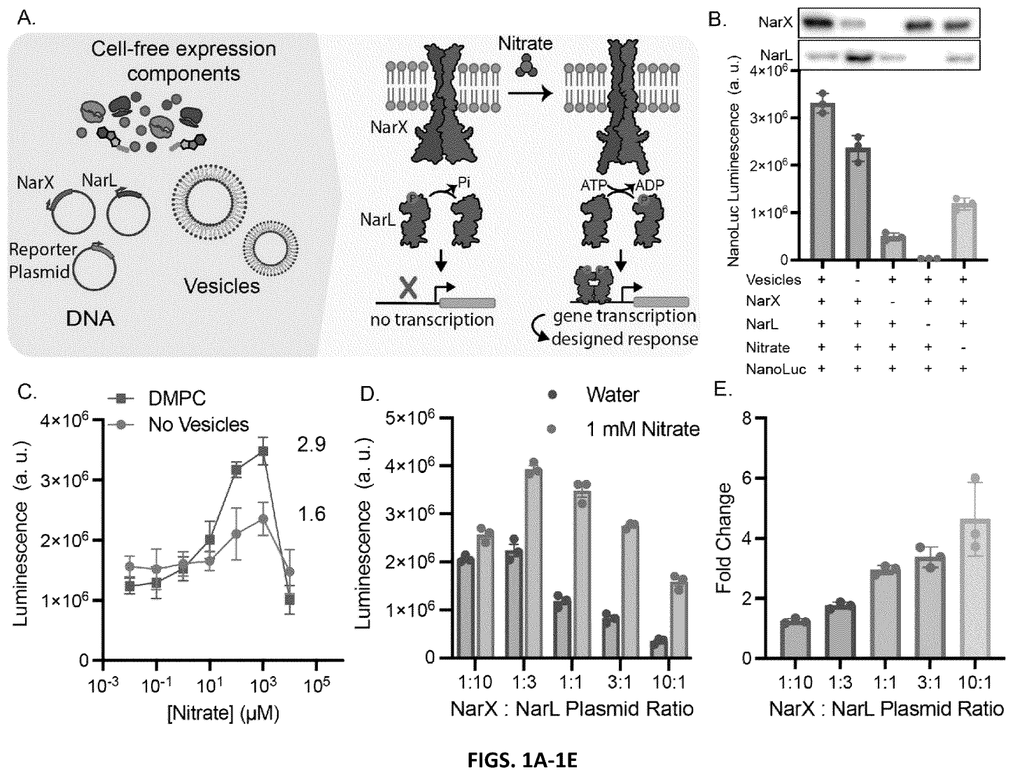

As a first step towards understanding how TCSs can be integrated into cell-free systems, we tested the functional expression of NarX and NarL. The analyte for this sensor, nitrate, is a known ground water contaminate (30), serves as a convenient input, and previous work has characterized and engineered NarL mutants and its cognate promoter (26), ensuring that downstream gene transcription is a result of cell-free expressed NarL. We assembled cell-free reactions containing the myTXTL system (31), three DNA templates, liposomes, and assessed the reactions in response to nitrate addition. Two templates contained genes for either NarX or a NarL mutant (26) under the control of T7 RNA polymerase. The third template served as a genetic reporter for TCS signaling and contained a reporter gene (nanoluciferase) under the control of a promoter to which NarL binds (32). Finally, we included 100 nm lipid vesicles composed of 1,2-dimyristoyl-sn-glycero-3-phosphocholine (DMPC) into which NarX could integrate (FIG. 1A). Upon addition of nitrate, NarX should phosphorylate NarL, which should then bind the reporter plasmid and induce downstream luciferase expression (FIG. 1A).

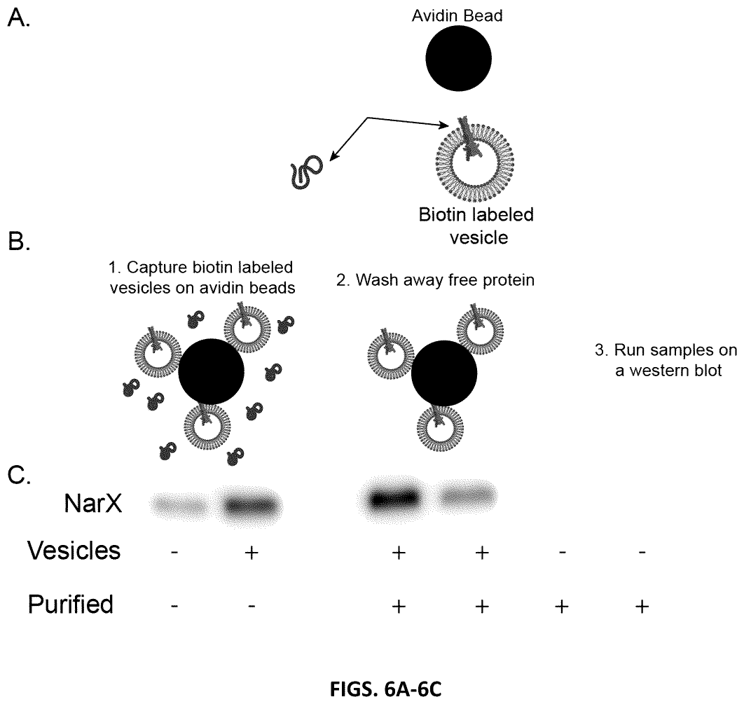

We first tested NarX-L function in our cell-free system by monitoring NarX and NarL expression and luciferase luminescence as a function of reaction components. We observed the highest luminescence when all components were present, about a 3-fold increase compared to reactions run in the absence of nitrate, indicating that nanoluciferase expression depends on both nitrate (FIG. 1B) and NarX and NarL expression. We confirmed NarX integration into vesicle membranes via immunoprecipitation (FIG. 6). When vesicles were removed from the reaction, we still observed luminescence, likely due to nonspecific binding of NarL to its cognate promoter and subsequent transcription when NarL is expressed at elevated levels. This non-specific interaction of NarL with the reporter plasmid is further supported by the dramatic reduction in luminescence upon removal of the NarL plasmid (FIG. 1B).

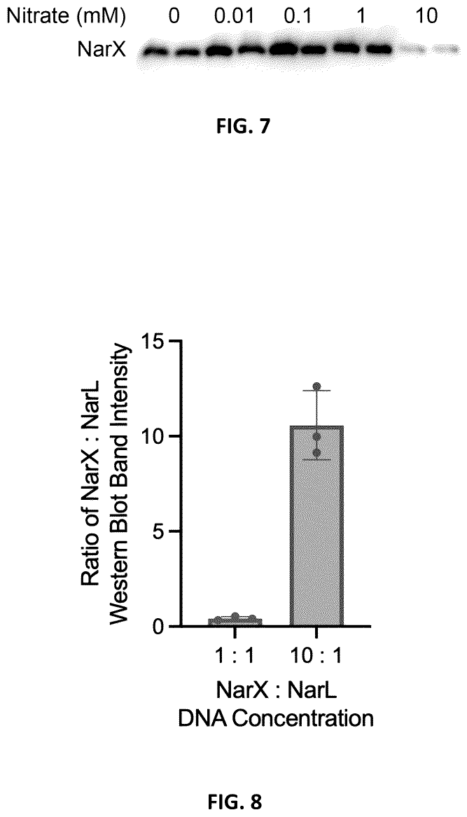

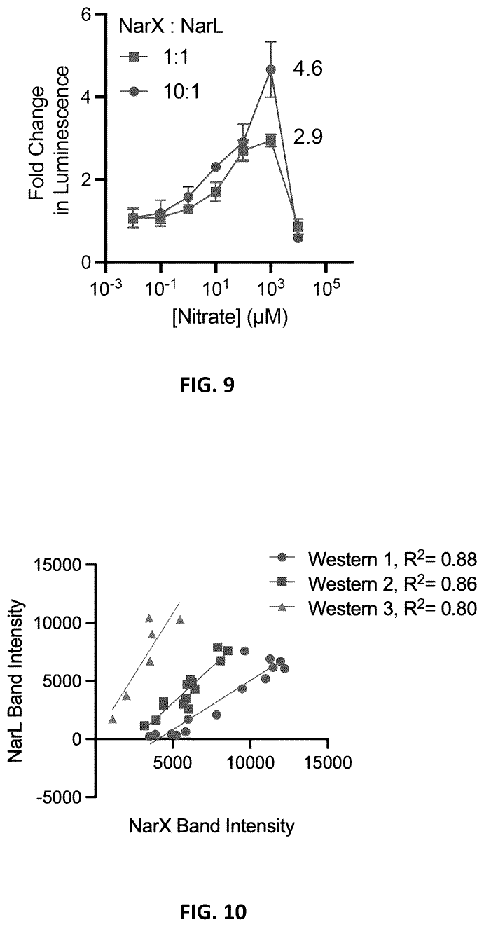

We then evaluated nitrate sensitivity and the role of membranes in our system by conducting a titration of nitrate with reactions containing either no vesicles or DMPC vesicles. In the absence of vesicles, we observed a 2-fold increase in luminescence indicating either that NarX may be cotranslationally inserting into native vesicles present in the extracts (33, 34) or there may be active, native NarX present in lysates (35). When DMPC vesicles were present, we observed a higher maximum luminescence, suggesting that NarX activity is enhanced by the synthetic vesicles, likely through cotranslational integration into the vesicle membranes (FIG. 1C). We determined our cell-free system allowed for detection of nitrate at levels as low as 10 μM, which is below the EPA limit (˜100 μM)(30). Our cell-free system lost sensitivity when nitrate concentrations were increased to 10 mM nitrate, as higher levels of nitrate inhibit protein synthesis (FIG. 7).

We then sought to tune sensor performance by altering the expression of NarX and NarL. In cell free systems, protein expression can be easily tuned by altering the amount of plasmid present in reactions (FIG. 8) (36). By retaining the total amount of NarX and NarL plasmid (6.6 nM), but altering the ratio of NarX and NarL plasmid from 1:10 to 10:1, we observed how expression impacted the expression of luciferase and the sensitivity of the sensor (FIG. 1D, E). Increasing the amount of NarL plasmid increased luminescence in the absence of nitrate, likely due to nonspecific binding of NarL to the promoter (FIG. 1D). When NarX plasmid concentration was increased relative to NarL, we observed a reduction in the overall signal but an increase in fold change, calculated as the ratio of luminescence in the presence of 1 mM nitrate to luminescence in the absence of nitrate (FIG. 1E, 9). These experiments demonstrate that the NarX-L can be functionally expressed in cell-free systems, and its performance can be tuned by altering the ratio of NarX and NarL plasmid.

### Modulation of NarX Activity Through the Tuning of Membrane Biophysical Features

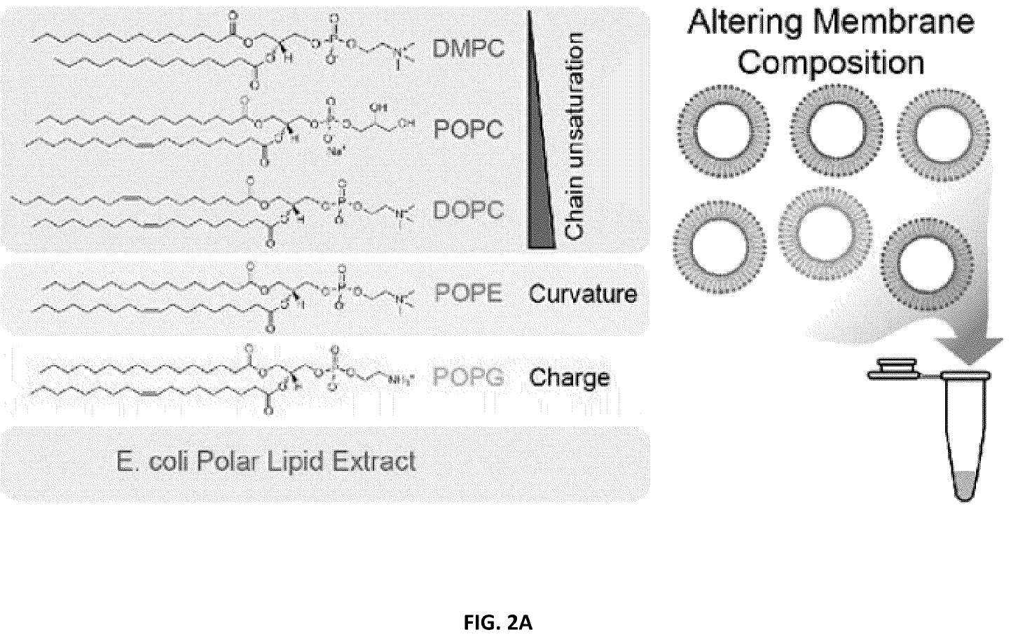

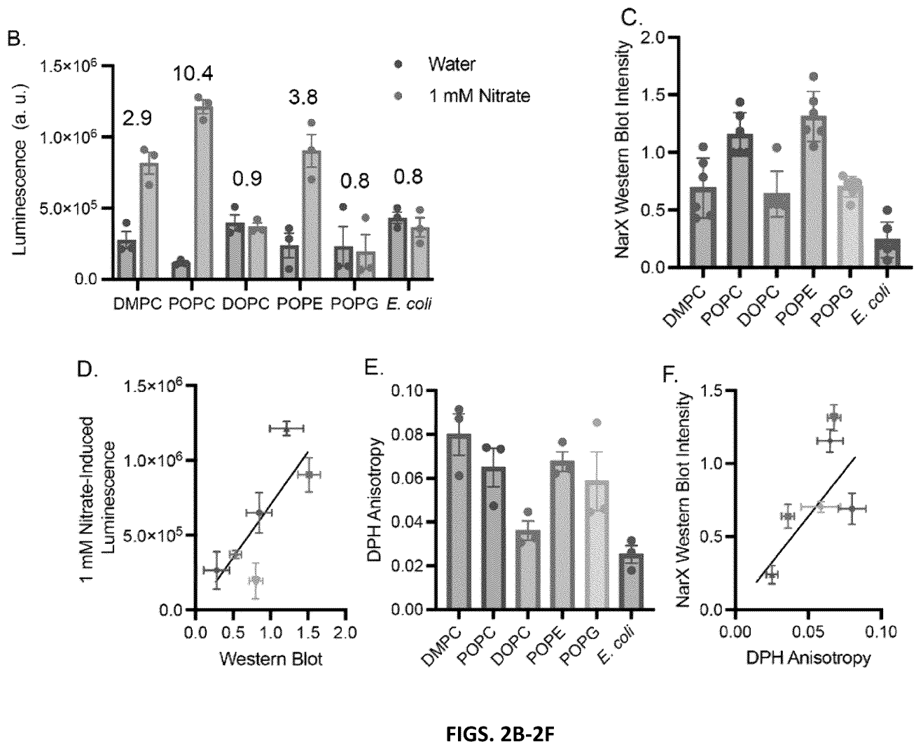

We next wondered if we could further tune sensor performance by modulating biophysical properties of the membranes present in the cell-free reaction. Membrane physical features, (such as lipid chain length and saturation, charge, curvature, and fluidity) have been shown to alter protein activity (37-40), as well as the cotranslational insertion and folding of membrane proteins (16, 41-43). To investigate the role of chain saturation on NarX-L performance, we assembled cell-free reactions with membranes composed of DMPC (14:0), POPC (18:1-16:0), and DOPC (18:1). These compositions should yield bilayer vesicles with membranes that are composed of lipids with two saturated acyl chains, one saturated and one unsaturated acyl chain, and two unsaturated chains, respectively. We further tested NarX-L activity in membrane systems with 30% POPE to investigate the impact of negative curvature lipid, 30% POPG to investigate the impact of membrane charge, and in 100% E. coli polar lipid extract, NarX's native membrane environment (FIG. 2A). We assembled cell-free reactions with 100 nm vesicles containing these different membrane compositions with and without 1 mM nitrate (Table 1). We found that using POPC membranes in cell-free reactions yielded the highest luminescence in the presence of nitrate and induced the highest fold-change in response to the addition of nitrate (FIG. 2B).

TABLE 1
Dynamic light scattering of vesicles compositions
used in FIG. 2. Presented is an average and standard
deviation of 2 independently prepared samples.

| | Diameter | | |
|---|---|---|---|
| | (nm) | SD (nm) | P.D.I. |
| 100% DMPC | 127.44 | 43.22 | 0.115 |
| 100% POPC | 128.14 | 66.41 | 0.269 |
| 100% DOPC | 124.71 | 62.77 | 0.253 |
| 30% POPE, 70% POPC | 118.24 | 47.02 | 0.158 |
| 30% POPG, 70% POPC | 112.91 | 61.28 | 0.295 |
| 100% E. coli Polar Lipid Extract | 127.19 | 50.47 | 0.157 |

We wondered if membrane composition impacted protein expression and thereby affected the cell-free sensor performance. To better understand how membranes affect protein expression, we performed western blots on our reactions to quantify NarX and NarL expression (FIG. 2C). We found that NarX expression is correlated with NarL and luciferase expression. Thus, we suspect that expression and proper folding of NarX likely enhances the yield of NarL and the downstream nanoluciferase expression in cell-free reactions (FIGS. 2D and 10). Further, improper membrane protein folding has been found to increase aggregation and truncation products (41, 44), and likely inhibits efficient expression of all protein components in the system. As a result of this relationship, we only considered NarX expression in our future analysis of the effect of membrane composition on NarX-L performance.

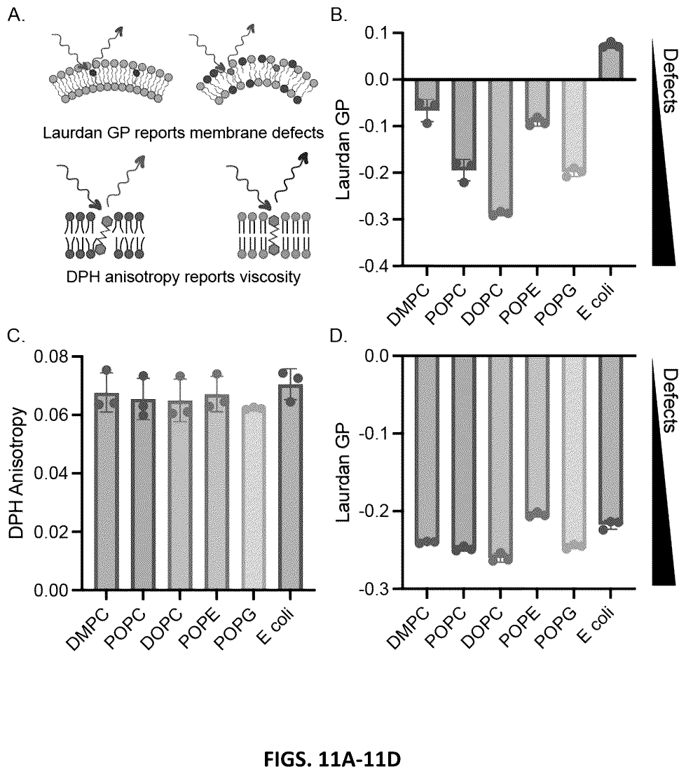

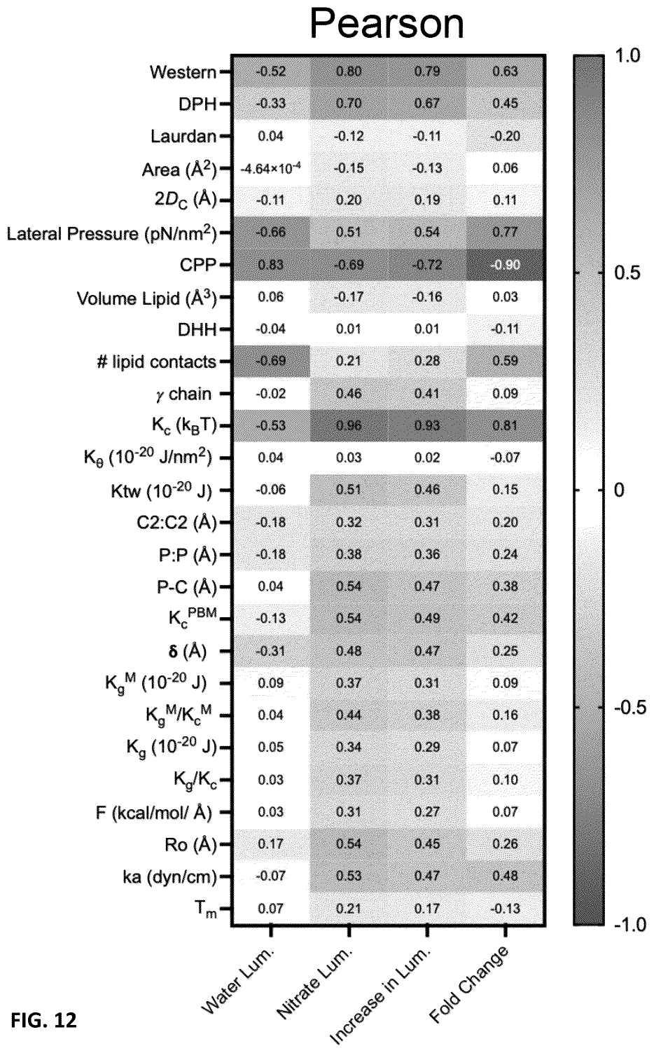

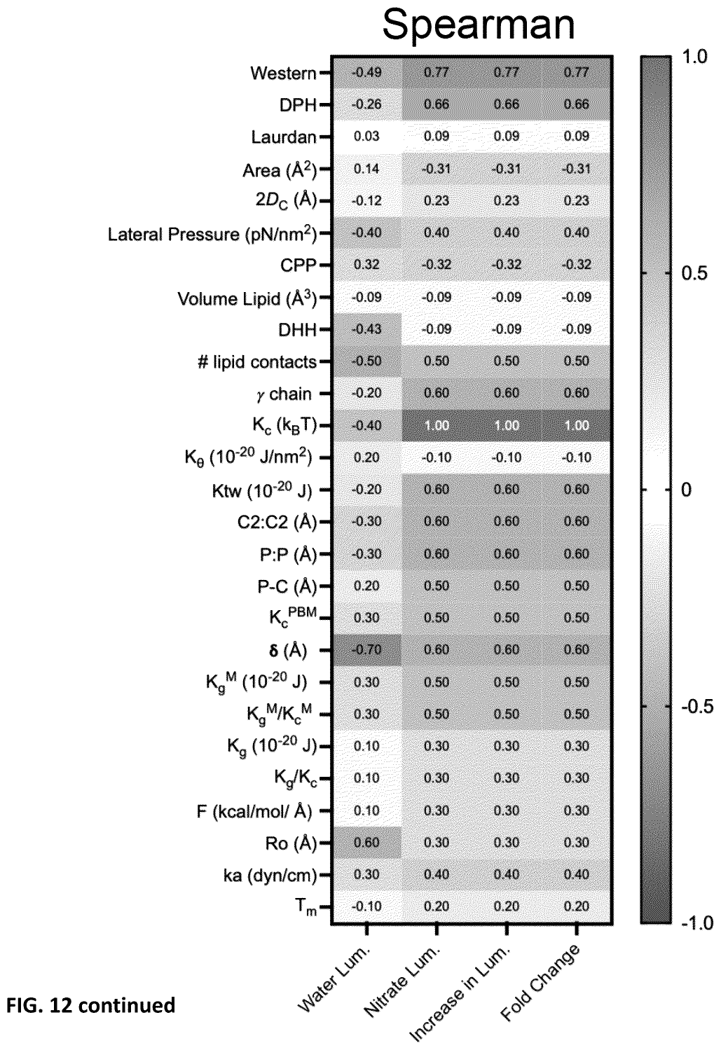

To better understand what properties of the membrane enable efficient protein expression, we measured membrane fluidity via DPH fluorescence anisotropy and lipid packing via Laurdan generalized polarization (GP) and compiled other lipid physical properties reported in literature (FIGS. 2E and 11, Table 2). Lipid properties are interrelated and often non-monotonic, thus making it difficult to directly relate a single membrane property to protein insertion and activity. To gain a picture of what properties may affect protein yield, we calculated Pearson's and Spearman's coefficients for all compiled membrane measurements to protein activity and expression (FIG. 12). Interestingly, we found that DPH anisotropy, which is used as a measure of membrane viscosity, positively correlated with NarX expression (FIG. 2E). Additionally, membrane lateral pressure, lipid neutral plane distance, and membrane transition temperature values compiled from literature also correlated with NarX expression. These features are affected by the structure of the hydrophobic region of the membrane and greatly affect membrane viscosity, further suggesting that membrane viscosity is a key feature for proper cotranslational insertion of NarX (FIG. 12).

TABLE 2
Lipid physical properties found in literature.

| | POPC | DOPC | DMPC | POPE | POPG | E. coli |
|---|---|---|---|---|---|---|
| Area (Å²) (1) | 64.3 | 67.4 | 60.6 | 56.6 | 66 | 56.8 |
| 2 D_{c} (A) (2) | 28.8 | 28.8 | 25.4 | 32.6 | 27.9 | 30 |
| Lateral Pressure | 517 | 480 | 442 | 456 | | |
| (pN/nm²) (3) | | | | | | |
| CPP (4) | 0.61 | 1.09 | 1 | 1.08 | 0.7 | |
| Lipid Volume | 1231.2 | 1304.5 | 1100 | 1176.8 | 1226.6 | 1186.6(1) |
| (A³) (3) | | | | | | |
| D_{HH} (Å) (2) | 37 | 36.7 | 35.3 | 43.4 | 38.5 | 40.8(1) |
| # of lipid contacts (5) | 2.75 | | | 1.5 | | 2.25 |
| γ chain (6) | 0.198 | 0.18(5) · | 0.247 | 0.188 | | |
| | | 221 | | | | |
| K_{c} (k_{B}T) (6) | 31.7 | 28.8 | 29.3 | 31.2 | 26.9 | |
| K_{θ} (10⁻²⁰ J/nm²) (6) | 5.52 | 6.4 | 4.02 | 8.04 | 6.15 | |
| K_{tw} (10⁻²⁰ J) (6) | 1.45 | 0.99 | 2.18 | 2.36 | 1.05 | |
| C2:C2 (Å) (6) | 27.52 | 27.37 | 24.54 | 30.67 | 26.88 | |
| P:P (Å) (6) | 38.54 | 38.35 | 35.65 | 41.55 | 37.49 | |
| P-C (Å) | 11.02 | 10.98 | 11.11 | 10.88 | 10.61 | |
| Head group | | | | | | |
| thickness (6) | | | | | | |
| K_{c}^{PBM} (6) | 9.5 | 9.71 | 5.75 | 10.78 | 5.67 | |
| δ (Å) (6) | 9.64 | 9.41 | 9.48 | 10.12 | 9.57 | |
| K_{g}^{M} (10⁻²⁰ J) (6) | −1.09 | −0.84 | −1.32 | 0.06 | −1.43 | |
| K_{g}^{M}/K_{c}^{M} (6) | −0.17 | −0.14 | −0.22 | 0.01 | −0.25 | |
| Kg (10⁻²⁰ J) (6) | −1.38 | −0.08 | −3.23 | 5.85 | −2.76 | |
| K_{g}/K_{c} (6) | −0.1 | −0.01 | −0.26 | 0.44 | −0.24 | |
| F (kcal/mol/Å) (6) | 0.0303 | 0.061 | −0.0225 | 0.2041 | 0.0036 | |
| Rₒ (Å) (6) | −315 | −1(5)392 | −47 | −2220 | | |
| Kₐ (dyn/cm) (6) | 280 | 290 | 210 | 260 | 180 | |
| Tm (7) | −9 | −17 | 24 | 25 | −2 | |

To investigate how membrane physiochemical interactions affect NarX activity, we made POPC vesicles and doped 10 mol % of an additional lipid into the membrane (FIG. 3A). POPC was chosen as the main membrane component as it was found to yield the largest fold change in luminescence upon the addition of nitrate (FIG. 2B). By only varying 10 mol % of the lipids in a given membrane composition and keeping the other 90 mol % consistent, we could ensure protein expression of NarX (and correspondingly NarL) remained constant across membrane compositions, thus allowing activity to be attributed to differences in protein conformational changes due to an altered membrane environment instead of total NarX-L protein present in each reaction (FIG. 3B). We first confirmed that these lipid compositions formed monodisperse liposomes (Table 3).

TABLE 3
Dynamic light scatting of vesicle compositions used in FIG. 3.
All vesicles are composed of 90% POPC and 10% of lipid
listed in the table. Presented is an average and standard
deviation of 2 independently prepared samples.

| | Diameter (nm) | SD (nm) | P.D.I. |
|---|---|---|---|
| DMPC | 129.61 | 60.54 | 0.218 |
| POPC | 128.14 | 66.41 | 0.269 |
| DOPC | 129.86 | 72.76 | 0.271 |
| POPE | 125.23 | 61.91 | 0.244 |
| POPG | 121.16 | 51.83 | 0.183 |
| E. coli | 128.33 | 47.69 | 0.209 |

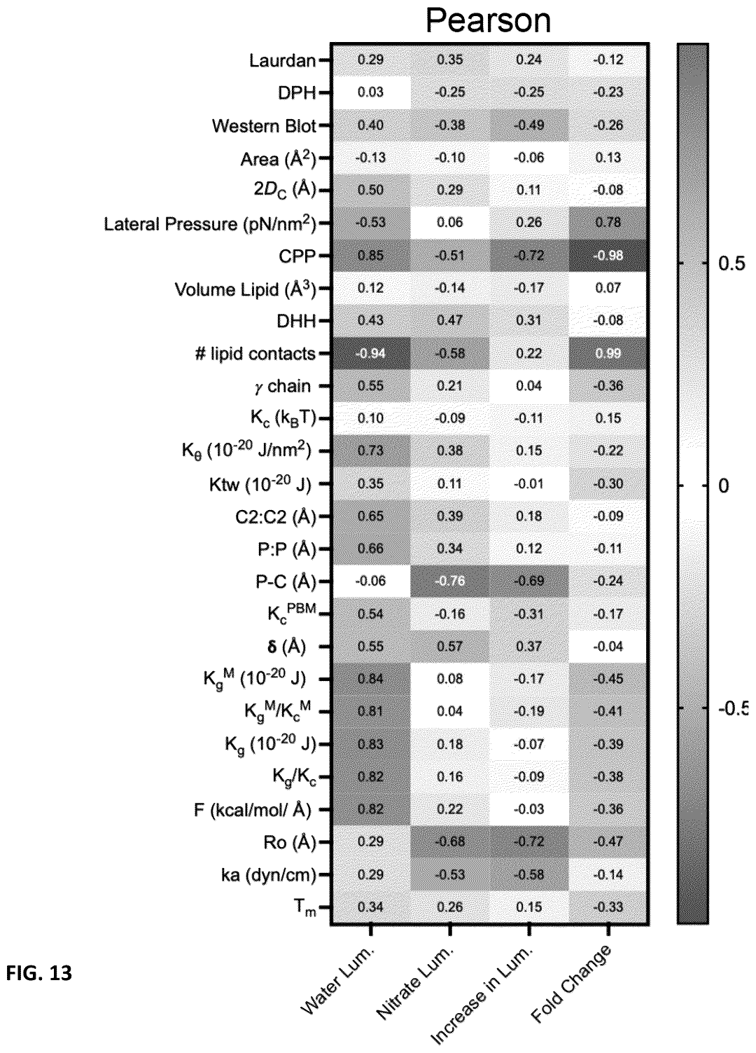

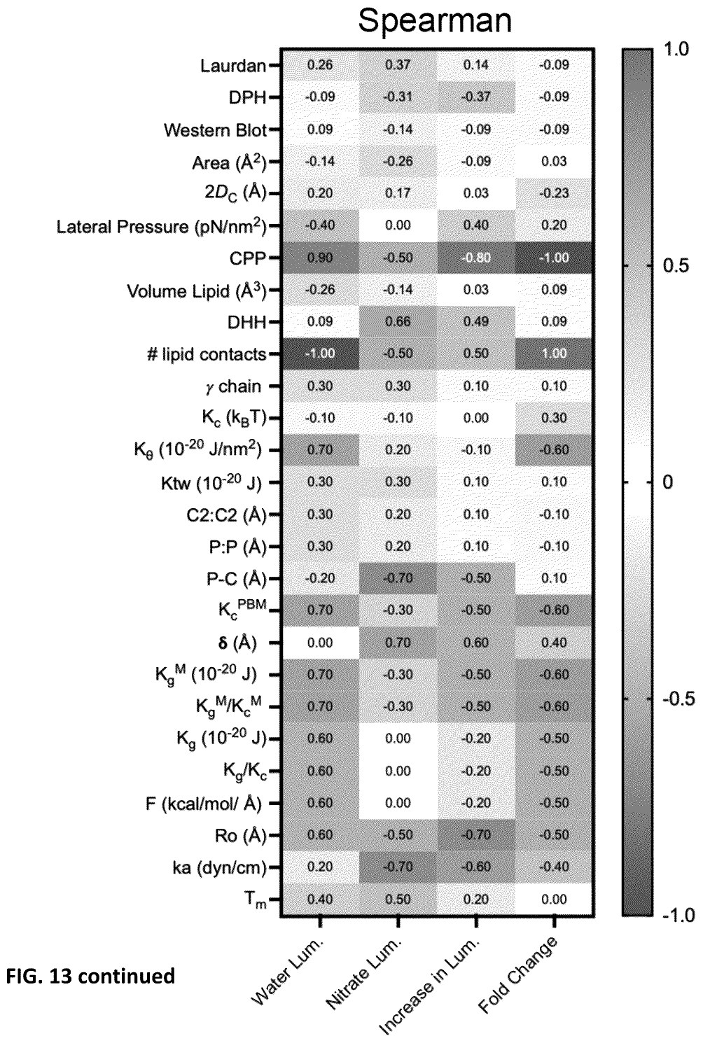

We again investigated the effect of chain saturation (DMPC, POPC, DOPC), charge (POPG), curvature (POPE), and E. coli native membrane lipids on NarX-L activity. POPC still yielded the best fold-induced luminescence, however, doping POPG into membranes yielded the largest increase in luminescence. Further, POPE yielded high luminescence in the presence of nitrate, but interestingly we observed an increase in luminescence in the absence of nitrate (FIG. 3C). This nitrate-independent increase in luminescence upon addition of POPE may suggest that negative lipid curvature stabilizes the “on” conformation of NarX (44). The high induction of luciferase expression in POPE and POPG membranes is unsurprising as PE and PG make up the majority of lipid headgroups in E. coli membranes (46). Adding E. coli polar lipid extract yielded a fold change in luminescence close to that of POPG, but a lower luminescence. DOPC still yielded a fold change less than 1, suggesting that increased fluidity may not support proper conformational changes required for induced phosphorylation of NarL and downstream expression upon binding to nitrate. To better understand what features of membranes may enhance sensor performance, we again performed Pearson and Spearman's correlations (FIG. 13). Physical parameters such as critical packing parameter (CPP), which describes the geometric shape of a lipid, and membrane thickness, as well as membrane mechanical properties, such as bending rigidity (Kc), were found to correlate with sensor performance (FIG. 3D, E, Table S2). Physical parameters compared to protein activity can be found in Table S2. Together, these experiments demonstrate that NarX-L expression and sensitivity can be tuned by altering the physiochemical properties of the membrane mimetic present in the cell-free reaction.

Expanding the Ligands that can be Sensed by the Membrane Bound Histidine Kinase NarX

Once we established that plasmid concentrations and membrane physiochemical properties could be leveraged to tune sensor function, we then wondered if we could generate new biosensors via protein engineering. Two-component systems are quite modular and amenable to protein engineering for the design of entirely new sensing and signaling systems in bacterial (24-26, 46) and mammalian systems (28). We therefore wondered if we could swap out the transmembrane and ligand binding domains of NarX, to generate entirely new cell-free sensors (25, 47-49). To accomplish this, we performed a sequence alignment on a subset of membrane bound histidine kinases, and selected candidates based on their alignment to NarX's HAMP domain, a helical region critical for transmitting ligand binding to kinase activity (FIG. 4A) (21, 47). From this analysis, we engineered copper, nickel, iron, and vancomycin sensing NarX chimeras. Specifically, we replaced the transmembrane and sensing domains, as well as the first half of the HAMP domain, of NarX with those from other proteins (NrsS, RssA, VanS, CusS), but retained the histidine kinase (FIG. 4B) (50-53). By swapping out the transmembrane and sensing domains, but retaining the kinase, we could leverage the same transcription factor and promoter to control downstream gene transcription in response to new ligands.

We identified several sensing domains that were active in response to the ligand of interest. Kinase and nanoluciferase expression were able to proceed upon addition of the nickel-, iron-, and vancomycin-sensing NarX chimera templates and their ligands. We confirmed that each ligand did not affect liposome stability (Table 4).

TABLE 4
Dynamic light scatting of 100% POPC vesicles in the
presence of ligands used in this study. Presented is an average
and standard deviation of 2 independently prepared samples.

| | Diameter (nm) | SD (nm) | P.D.I. |
|---|---|---|---|
| 1 mM Nitrate | 123.26 | 64.93 | 0.278 |
| 1 mM Nickel | 123.27 | 54.07 | 0.192 |
| 100 μM Iron | 118.62 | 43.63 | 0.135 |
| 1 mM Vancomycin | 124.88 | 54.32 | 0.189 |

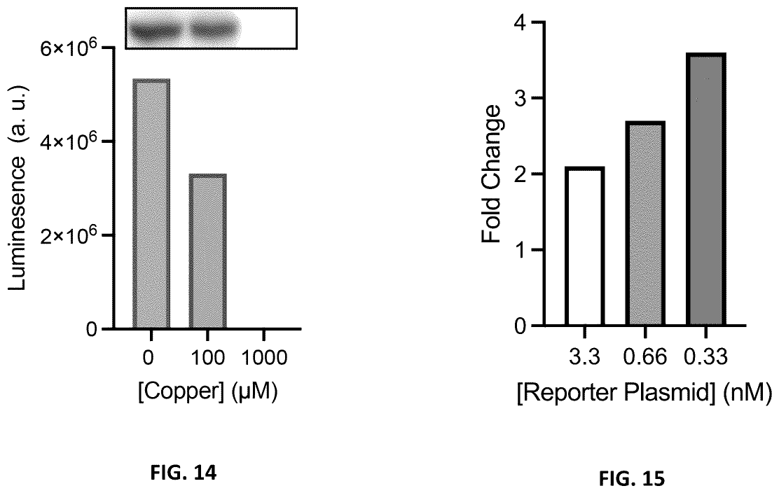

Unfortunately, the addition of copper inhibited cell-free protein expression (FIG. 14). Using plasmid concentrations, which enabled functional NarX sensing, yielded a functional nickel sensor, but no gene expression was observed for the iron and vancomycin sensors in response to each corresponding ligand. We tuned down the concentration of the reporter plasmid by 90% to reduce background expression of nanoluciferase and enhance the specificity of the sensors (FIG. 15). We then expressed proteins in the presence of DMPC, POPC, and DOPC vesicles (FIG. 4C, D, E). Upon reduction of reporter plasmid, we generated functional sensors in response to nickel and iron. Interestingly, each sensor performed differently in each membrane, suggesting that each protein requires specific membrane physiochemical properties for proper function. The vancomycin sensor did not induce an increase in luciferase expression in any of the membrane compositions tested in response to vancomycin. However, luminescence for the vancomycin sensor was highest across all conditions (FIG. 4E), suggesting that the chimera may be locked into an “on” conformation, perhaps due to destabilizing the HAMP domain (44). These results demonstrate how protein and membrane engineering can be used to readily generate new cell-free receptors by tapping into the large repertoire of known sensing domains of transmembrane proteins, and underscores the important relationship between membrane physical properties, protein sequence, and function.

Once we established that we could successfully sense multiple ligands, we wondered if we could co-express multiple kinases into vesicles to sense more than one ligand at a time. To test this, we chose to co-express NarX and the nickel-sensing chimera as they both exhibit activity in POPC membranes. We first established the ability of wild-type NarX and the nickel-sensing chimera to specifically turn ‘on’ in response to nitrate and nickel (FIG. 4F). Once specificity was confirmed, we co-expressed wild-type NarX and the nickel-sensing chimera into POPC vesicles in the presence of water, 1 mM nickel, 1 mM nitrate, or 1 mM nickel and 1 mM nitrate (FIG. 4G). We found that upon co-expression, we could sense multiple ligands. Interestingly, we observed the highest luminescence in the presence of both ligands, perhaps because both kinases were able to initiate transcription, leading to increased luciferase expression (FIG. 4H). Integrating multiple receptors into synthetic membranes together with the design of distinct genetic reporters could lead to synthetic vesicles capable of multiplexed sensing and response to many diverse stimuli. Priming cell-free expressed two-component systems for real world biosensing.

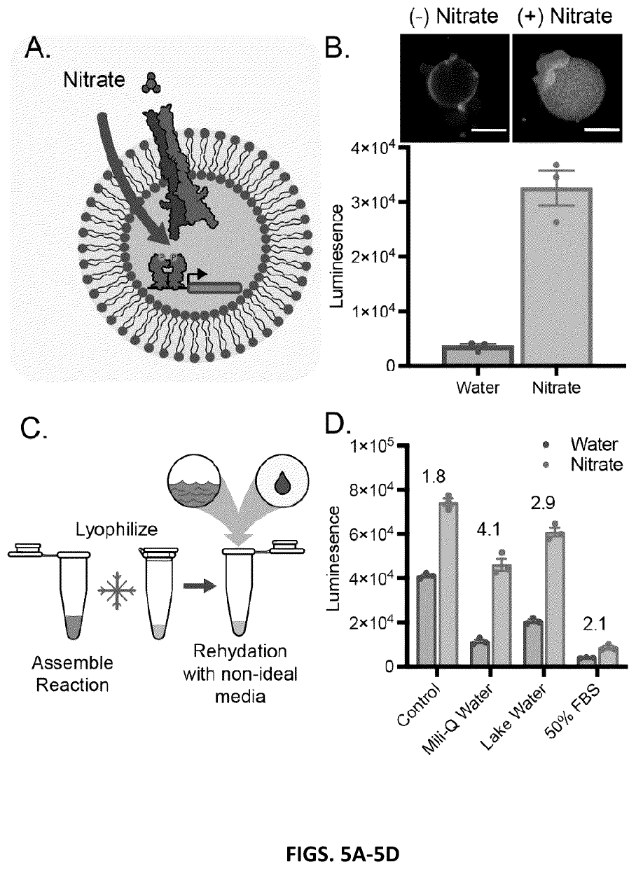

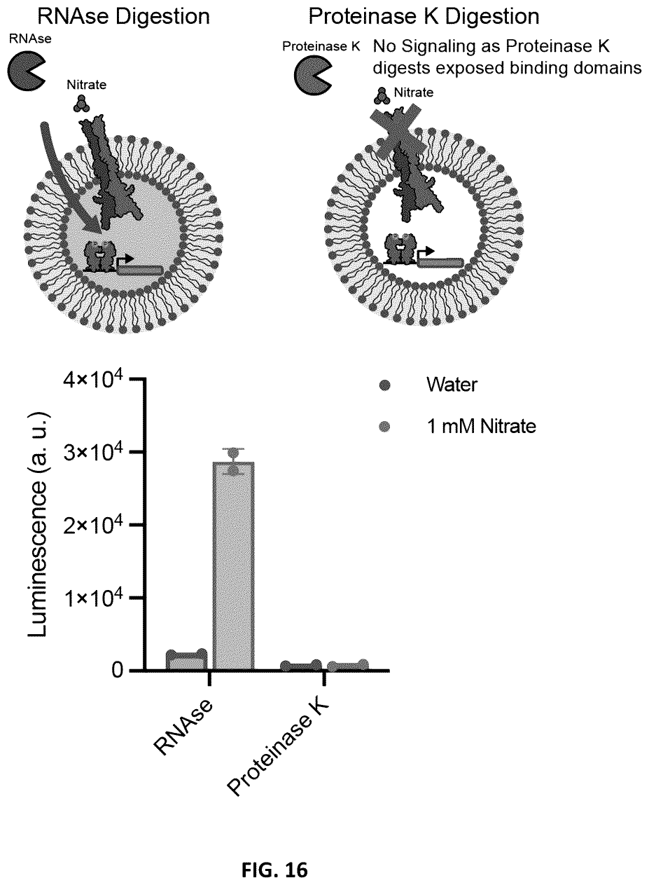

Over the last decade, many promising cell-free systems have been developed, enabling the delivery and sensing of a wide array of ligands in non-ideal environments. Cell-free systems have been encapsulated and administered to mice (47), lyophilized and used in resource limited settings (6, 7), and integrated into wearable materials to sense environmental contaminants (8). Towards the goal of creating cell-mimetic particles which can sense environmental stimuli in complex environments, such as the body, we encapsulated our cell-free system into giant unilamellar vesicles using the emulsion transfer method (FIG. 5A). We encapsulated cell-free extract containing plasmids encoding NarX, NarL, and the downstream protein (luciferase or mEGFP) into membranes composed of 1 POPC: 1 cholesterol and a rhodamine conjugated lipid. We hypothesized that by encapsulating cell-free reaction components into vesicles, NarX could insert into membranes and initiate signaling in response to exogenously added nitrate. Vesicles encapsulating the reactions were incubated at 30° C. for 16 hours with RNase, to prevent transcription outside of the vesicles, and in the presence or absence of exogenously added 1 mM nitrate. We observed that the sensor remained functional and was able to sense exogenous nitrate via microscopy and a bulk plate reader assay (FIG. 5B). To further confirm that NarX properly inserted into membranes, we repeated this experiment, adding Proteinase K to the outside of vesicles. Upon addition of Proteinase K, signaling was inhibited, likely because Proteinase K digested external nitrate binding domains (FIG. 16). When incubated with RNAse, the encapsulated reaction proceeds in a nitrate dependent manner. Combined, these data demonstrate that the NarX-L system can be functionally encapsulated into liposomes, which may allow for transmembrane sensing to be utilized in complex aqueous environments.

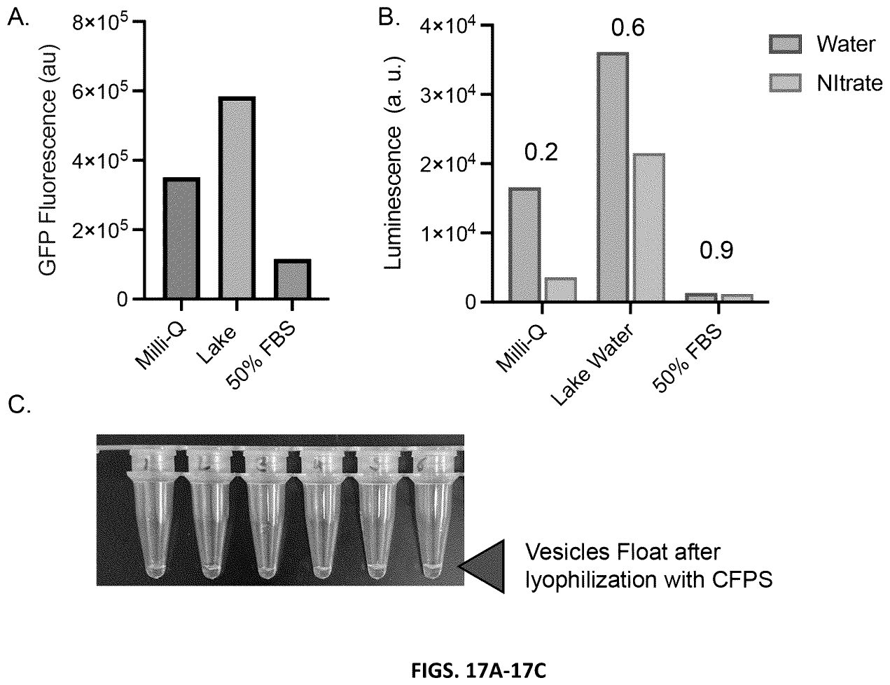

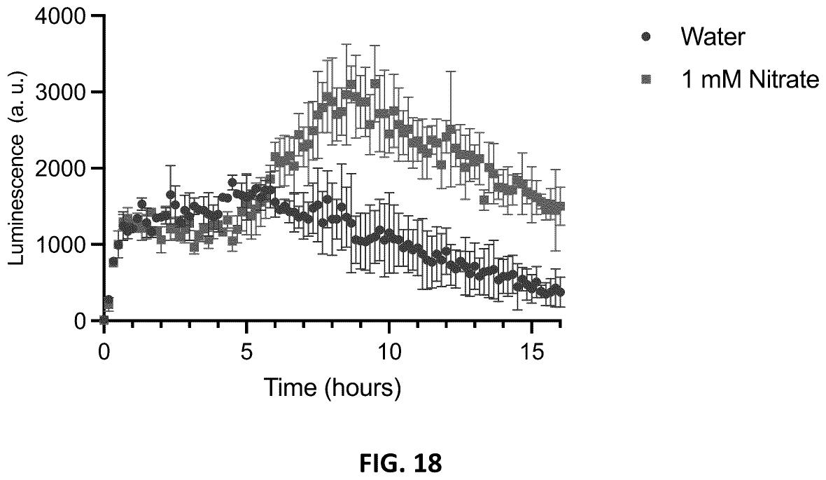

To demonstrate our system's ability to be deployed out of the lab to sense ligands in non-ideal matrices, we lyophilized reactions and tested their ability to sense 100 μM nitrate, approximately the EPA limit of nitrate (30), when rehydrated with lab-grade Milli-Q water, water from Lake Michigan, and fetal bovine serum (FBS) (FIG. 5C). As a control, we flash froze reactions and allowed them to proceed without lyophilization. We found that lyophilizing vesicles and cell-free components separately produced better results. In reactions with vesicles present during lyophilization, vesicles were found to often float to the top, likely inhibiting membrane-bound kinase expression, and producing low inducible signal (FIG. 17). To remediate this, we lyophilized vesicles and reactions separately. When rehydrating reactions, we rehydrated vesicles with water and then mixed the rehydrated vesicles, lyophilized cell-free components, and rehydration media (water, lake water, or FBS) with or without 100 μM nitrate. We then allowed reactions to proceed at 30° C. We found that we could sense 100 μM nitrate in all conditions, however the expression and fold change in luminescence varied in each rehydration media, likely due to different interactions with membranes and transcription/translation machinery (FIG. 5D). To deploy biosensors out of the lab, one must understand how quickly they are able to sense a ligand. To accomplish this, we read luminescence over time and found that we could differentiate a positive from a negative signal in 6.5 hours (FIG. 18). Combined, these data demonstrate the ability of cell-free expressed bacterial two-component systems to be integrated and functional in contexts amenable for real-world deployment. We furthermore envision that these systems could be leveraged to engineer targeted drug delivery systems or for point-of-use biosensing.

### DISCUSSION

Here, we show that the bacterial TCS, NarX-L, can be functionally reconstituted into a cell-free system. Leveraging the tunability of cell-free systems, we demonstrate how reaction composition and membrane design may be used to tune the performance of a model two component system, NarX-L. Employing rational protein design we generate new nickel and iron sensors and explore how transmembrane sequences may require membranes with different physiochemical properties for optimal function. Much work has been done characterizing and engineering membrane bound TCS's in both prokaryotic and eukaryotic systems (21, 26, 28, 46, 48); and we now show that these systems can be reconstituted in vitro into synthetic membranes which may serve as a platform to both probe biophysical interactions between protein and membrane components and enable new membrane-based biosensors and therapeutics.

Characterizing how membrane-protein interactions affect membrane protein folding and activity is integral to uncovering how such interactions may contribute to disease or be leveraged in biotechnologies (9, 56, 57). Toward this goal, in vitro systems have been used to characterize purified protein activity and cotranslational folding of membrane proteins. Previous work has explored how membrane composition and mechanical properties affect protein integration and folding into synthetic membranes (16, 42). Separately, membrane proteins have been extracted from cells and studied in synthetic membranes. This process allows for the study of specific membrane properties on protein function and has enabled a better understanding of energy generation (51), lipid sensing (40), and cellular signaling (39, 55). However, to the best of our knowledge, we are the first to demonstrate and characterize the folding and activity of a cell-free expressed transmembrane protein-to-genetic reporter signaling pathway. Such a system enables the characterization of protein folding and function in one pot and does not require protein purification and reconstitution.

Through this work, we found that membrane viscosity greatly impacts protein insertion and folding. Interestingly, we found that PE and PG lipids, which are the most abundant headgroups found in E. coli, NarX's native membrane, allowed for the highest luminescence in response to nitrate. Furthermore, the relationship between membrane properties and activity for our chimeric iron sensor agrees with previously reported in vivo data demonstrating that RssA is inhibited by saturated fatty acids (52). We showed that NarX is inserted into membranes and activity correlates with the critical packing parameter of our synthetic membranes (FIGS. 7, 16, 3). However, from our studies we cannot exclude the possibility that a fraction of NarX is improperly inserted or only associated with membranes. Moving forward, performing further protein structure-based experiments to gain a more detailed understanding of how lipid-protein interactions specifically affect NarX function could allow for a more complete understanding of how the lipid environment affects TCS signaling (60). Together, this data demonstrates that this platform may approximate activity in membranes with similar biophysical properties and could provide a route to assess the relationship between membrane physiochemical properties and membrane protein folding and activity in high-throughput. Further, our results demonstrate that protein activity is greatly affected by the membrane physiochemical environment and that proteins with different sequences require different lipid environments for proper function.

Cell-free systems have been shown to be powerful platforms to build biosensors (6-8, 53) and therapeutics (3, 5). Over the last 20 years, membranes have been used to encapsulate cell-free systems, towards the goal of engineering an artificial cell (1, 9). Such encapsulation systems have been shown to enable prolonged protein expression (10), release of cargo in response to environmental cues (11, 17), as well as protection from degradation (12). However, to date such artificial cellular systems have relied on the nonspecific transport of materials across the membrane to initiate a response. This approach limits the types of signals which can be sensed to those that passively cross the bilayer membrane and when non-specific pores are used to improve molecular transport, they risk leakage of encapsulated components required for function. Together the limited design of membranes that effectively utilize transmembrane proteins has vastly limited the potential applications of membrane-augmented cell-free systems to date.

The recapitulation of a transmembrane protein-to-genetic reporter signal transduction mechanism opens the door to exploring a wide range of membrane proteins as sensors in cell-free systems. Extending this platform beyond biophysical characterization of transmembrane signaling could enable the creation of new membrane-based biosensors and therapeutics. By incorporating genetic parts already developed for bacterial two-component systems (21, 24, 26), we believe that transmembrane signaling in response to a wide array of ligands including metals, small molecules, and proteins is within reach. Further, novel receptor systems have been developed in cellular systems to enable cells to sense and respond to new ligands (55-60), and their integration into cell-free systems could greatly expand sensing capabilities of cell-mimetic systems. Engineering materials capable of sensing and responding to stimuli would combine the responsiveness of cell-based therapeutics with the tunability of cell-free systems, drastically improving our ability to engineer targeted therapeutic delivery systems and new membrane-based materials.

### Materials and Methods

### Materials

1,2-dimyristoyl-sn-glycero-3-phosphocholine (DMPC), 1-palmitoyl-2-oleoyl-glycero-3-phosphocholine (POPC), 1,2-dioleoyl-sn-glycero-3-phosphocholine (DOPC), 1-palmitoyl-2-oleoyl-sn-glycero-3-phosphoethanolamine (POPE), 1-palmitoyl-2-oleoyl-sn-glycero-3-phospho-(1′-rac-glycerol) (sodium salt) (POPG), E coli Polar Lipid Extract, Cholesterol, and 1,2-dioleoyl-sn-glycero-3-phosphoethanolamine-N-(7-nitro-2-1,3-benzoxadiazol-4-yl) 1,2-dioleoyl-snglycero-3-phosphoethanolamine-N-(lissamine rhodamine B sulfonyl) (18:1 Rhodamine) were purchased from Avanti Polar Lipids. Cell-extract (myTxTl) was purchased from Arbor Bio Sciences. gBlocks and primers were ordered from Integrated DNA technologies and DNA was amplified and assembled using enzymes from Thermo Fisher. Ligands sodium nitrate, nickel (II) sulfate hexahydrate, iron (III) chloride anhydrous, vancomycin hydrochloride, and copper (II) sulfate pentahydrate were obtained from Thermo Fisher. NanoGlo luciferase was purchased from Promega.

### Plasmid Design and Construction

DNA encoding NarX, NarL, YdfJ promoter, and nanoluciferase were received as gifts from Michael Jewett and were recloned into cell-free backbones using Gibson Assembly. gBlocks encoding kinase chimeras and primers for cloning were ordered from Integrated DNA technologies. Enzymes and buffers required for PCR and cloning were purchased from Thermo Fisher. Iron sensing histidine kinase RssA (Serratia marcescens) sequence was taken from Uniprot Entry Q8GP19 (51). The copper sensing histidine kinase (E. coli) sequence was taken from Uniprot Entry P77485 (50). The vancomycin sensing histidine kinase VanS (Enterococcus faecium) sequence was taken from Uniprot Entry Q06240 (52). The nickel sensing histidine kinase NrsS (Synechocystis sp.) sequence was taken from Uniprot Entry Q55932 (53). All DNA sequences can be found in Table 5.

TABLE 5
DNA sequences used in this study. All genes were
placed under the control of a T7 promoter, except
nanoluciferase and GFP. These constructs were under
the control of the PydfJ115 promoter.
The promoter sequence is listed in with the gene.

| Plasmid/Gene | Sequence |
|---|---|
| NarX | ATGCTTAAACGTTGTCTCTCTCCGCTCACCCTGGTTAATCAGGTT |
| (SEQ ID NO: | GCGCTTATTGTGTTGCTTTCTACTGCTATTGGACTGGCAGGGATG |
| 1) | GCGGTTTCTGGCTGGCTGGTGCAAGGCGTTCAGGGCAGCGCCCA |
| | TGCGATCAACAAAGCGGGATCGCTGCGCATGCAAAGTTACCGTC |
| | TGTTGGCGGCAGTGCCATTAAGCGAGAAAGACAAGCCCTTAATT |
| | AAAGAGATGGAACAAACGGCATTTAGCGCCGAGTTGACTCGAG |
| | CAGCAGAACGAGACGGACAACTGGCGCAATTACAGGGTTTACA |
| | AGATTACTGGCGTAATGAACTGATCCCTGCGCTGATGCGTGCAC |
| | AAAACCGAGAAACGGTGTCAGCGGATGTCAGCCAGTTTGTTGCC |
| | GGGCTTGATCAACTGGTATCTGGTTTTGACCGCACCACGGAAAT |
| | GCGCATCGAGACAGTGGTACTGGTCCATCGGGTAATGGCGGTAT |
| | TTATGGCACTTTTACTGGTGTTCACTATTATCTGGTTGCGGGCGC |
| | GACTGCTACAACCGTGGCGGCAACTGCTGGCAATGGCGAGTGC |
| | CGTCAGTCATCGCGATTTTACCCAACGCGCAAACATCAGCGGGC |
| | GCAACGAAATGGCGATGCTTGGAACTGCGTTGAACAATATGTCT |
| | GCAGAACTGGCCGAAAGTTATGCCGTACTTGAGCAGCGGGTTCA |
| | GGAGAAAACCGCCGGGCTGGAGCATAAAAATCAGATCCTCTCT |
| | TTTTTATGGCAGGCTAACCGCCGTTTGCATTCCCGCGCCCCGCTG |
| | TGTGAACGCCTGTCACCTGTACTCAACGGCTTACAGAATTTAAC |
| | CCTGCTACGTGATATCGAATTGCGGGTGTATGACACTGATGATG |
| | AAGAGAATCATCAGGAGTTTACCTGCCAGCCAGATATGACTTGT |
| | GATGATAAAGGCTGCCAGCTCTGCCCGCGCGGCGTATTACCCGT |
| | TGGTGATCGCGGCACGACCCTGAAGTGGCGGCTGGCTGACTCTC |
| | ATACGCAGTACGGTATTTTGCTGGCGACCCTGCCACAGGGGCGT |
| | CATCTTAGCCATGATCAACAACAACTGGTGGATACCCTGGCTGA |
| | ACAACTCACCGCCACGCTGGCGCTGGATCGCCATCAGGAACGTC |
| | AGCAACAGTTGATCGTGATGGAAGAGCGTGCCACCATTGCGCG |
| | CGAACTGCATGATTCTATTGCCCAATCTCTCTCTTGCATGAAGAT |
| | GCAGGTGAGTTGTTTACAGATGCAGGGCGATGCGCTGCCAGAA |
| | AGCAGCCGCGAACTGTTAAGTCAGATCCGTAACGAACTGAATG |
| | CATCCTGGGCGCAGTTGCGTGAATTGCTCATCACATTCCGCTTG |
| | CAGCTCACCGAGCCTGGATTACGTCCGGCGCTGGAGGCGAGTTG |
| | CGAAGAGTACAGCGCCAAATTTGGCTTCCCGGTGAAGCTGGATT |
| | ATCAATTGCCGCCTCGCCTGGTGCCTTCGCATCAGGCAATCCAC |
| | TTGTTGCAAATTGCCCGTGAGGCATTAAGTAACGCCCTCAAACA |
| | TTCGCAAGCGAGTGAAGTCGTGGTGACGGTGGCGCAAAACGAT |
| | AATCAGGTCAAACTGACCGTCCAGGATAACGGCTGCGGCGTGC |
| | CTGAAAATGCCATCCGCAGCAATCACTACGGCATGATAATAATG |
| | CGCGATCGTGCGCAAAGTTTACGAGGCGATTGCCGCGTCCGCCG |
| | TCGTGAATCAGGTGGCACCGAAGTGGTGGTCACCTTTATTCCCG |
| | AAAAAACTTTCACAGACGTCCAAGGAGATACCCATGAGGGAGG |
| | AGGAAGCGAGCAGAAACTCATCTCTGAAGAGGATCTGTAA |
| NarL | ATGAGTAATCAGGAACCGGCTACTATCCTGCTGATTGACGATCA |
| (SEQ ID NO: | CCCGATGCTGCGAACTGGCGTAAAACAGCTTATCAGTATGGCAC |
| 2) | CAGATATCACCGTGGTTGGCGAAGCGAGTAATGGCGAACAGGG |
| | TATTGAACTGGCGGAGTCTCTTGATCCCGATCTGATCCTGTTAG |
| | ATCTCAATATGCCCGGCATGAACGGTCTGGAAACGCTGGATAAA |
| | CTGCGCGAAAAGTCCCTCTCAGGGCGCATTGTGGTATTCAGCGT |
| | CTCTAACCATGAAGAAGATGTGGTCACCGCACTGAAACGCGGC |
| | GCGGATGGCTATCTGTTAAAAGATATGGAACCGGAAGATCTGCT |
| | GAAAGCATTGCATCAGGCAGCTGCTGGCGAAATGGTATTAAGC |
| | CCTGATATCCTTAAACGTCTGCAAGAAATCCAATTTGAGCGGAT |
| | GAAAAAGCAGCGCAATGAGACGCAGCTGACAGAAAAGGAAGT |
| | CATTGTTCTAAAAGCAATTGCTAAAGGTCTTAAAAGCAAAGCGA |
| | TTGCCTTTGATTTGGGCGTCTCTGAGCGAACAGTAAAGTCCAGA |
| | TTAACGTCCATTTACAATAAATTAGGCGCGAATTCAAGAACTGA |
| | GGCAGTAACGATTGCCATGCAAAAAGGTATTCTGACAATAGAC |
| | AACggAGGAGGAAGCGATTACAAGGATGACGACGATAAGTAA |
| PYdfJ115_ | ACTGCATATTTGAAAATTGCCCAAACGTACATGCCCGAATGTAC |
| Nanoluc | GTTTTTTTCATTTCATTGTCAACTACAATGAGAAAGAATGTGATC |
| (SEQ ID NO: | AAGCAATGTGTTGAAAGGAGATTATCACGTCGACTCTCGAGTGA |
| 3) | GATTGTTGACGGTACCGTATTTTGGATCTAGGAGGAAGGATCTA |
| | TGGTGTTTACGCTGGAGGATTTCGTCGGTGACTGGCGTCAGACA |
| | GCTGGGTATAACCTTGACCAGGTACTTGAACAAGGCGGCGTTTC |
| | CAGCTTATTTCAAAATCTGGGGGTGTCTGTCACACCAATTCAGC |
| | GCATTGTCTTGTCTGGGGAGAATGGTCTTAAAATTGATATTCAT |
| | GTTATCATCCCTTACGAAGGTTTGTCCGGTGACCAAATGGGACA |
| | AATCGAGAAAATTTTCAAAGTGGTTTATCCAGTGGATGATCATC |
| | ACTTTAAAGTCATTTTACACTATGGTACGCTGGTAATCGACGGT |
| | GTGACACCGAACATGATTGATTATTTCGGGCGTCCGTATGAAGG |
| | AATCGCCGTTTTTGATGGGAAGAAAATCACCGTAACAGGTACTC |
| | TGTGGAACGGAAACAAAATCATCGACGAACGCTTGATTAACCC |
| | CGATGGTAGTTTGTTGTTCCGTGTTACGATTAACGGAGTTACGG |
| | GTTGGCGCCTTTGTGAGCGTATCTTGGCCGGAAGCGGAAGCGAG |
| | CAGAAACTCATCTCTGAAGAGGATCTGTAA |
| PYdfJ115_ | ACTGCATATTTGAAAATTGCCCAAACGTACATGCCCGAATGTAC |
| GFP | GTTTTTTTCATTTCATTGTCAACTACAATGAGAAAGAATGTGATC |
| (SEQ ID NO: | AAGCAATGTGTTGAAAGGAGATTATCACGTCGACTCTCGAGTGA |
| 4) | GATTGTTGACGGTACCGTATTTTGGATCTAGGAGGAAGGATCTA |
| | TGAGCAAAGGAGAAGAACTTTTCACTGGAGTTGTCCCAATTCTT |
| | GTTGAATTAGATGGTGATGTTAATGGGCACAAATTTTCTGTCCG |
| | TGGAGAGGGTGAAGGTGATGCTACAAACGGAAAACTCACCCTT |
| | AAATTTATTTGCACTACTGGAAAACTACCTGTTCCGTGGCCAAC |
| | ACTTGTCACTACTCTGACCTATGGTGTTCAATGCTTTTCCCGTTA |
| | TCCGGATCACATGAAACGGCATGACTTTTTCAAGAGTGCCATGC |
| | CCGAAGGTTATGTACAGGAACGCACTATATCTTTCAAAGATGAC |
| | GGGACCTACAAGACGCGTGCTGAAGTCAAGTTTGAAGGTGATA |
| | CCCTTGTTAATCGTATCGAGTTAAAGGGTATTGATTTTAAAGAA |
| | GATGGAAACATTCTTGGACACAAACTCGAGTACAACTTTAACTC |
| | ACACAATGTATACATCACGGCAGACAAACAAAAGAATGGAATC |
| | AAAGCTAACTTCAAAATTCGCCACAACGTTGAAGATGGTTCCGT |
| | TCAACTAGCAGACCATTATCAACAAAATACTCCAATTGGCGATG |
| | GCCCTGTCCTTTTACCAGACAACCATTACCTGTCGACACAATCT |
| | GTCCTTTCGAAAGATCCCAACGAAAAGCGTGACCACATGGTCCT |
| | TCTTGAGTTTGTAACTGCTGCTGGGATTACACATGGCATGGATG |
| | AGCTCTACAAATAA |
| NarX-CusS | ATGGTGAGCAAACCGTTTCAGCGTCCGTTTAGCCTGGCAACCCG |
| Chimera | TCTGACCTTTTTCATTTCACTGGCAACCATTGCAGCATTTTTCGC |
| (SEQ ID NO: | ATTTGCATGGATTATGATCCATAGCGTGAAAGTGCATTTTGCCG |
| 5) | AACAGGATATTAACGACCTGAAAGAAATTAGCGCAACCCTGGA |
| | ACGTGTTCTGAATCATCCTGATGAAACCCAGGCACGTCGTCTGA |
| | TGACCCTGGAAGATATTGTTAGCGGTTATAGCAATGTTCTGATT |
| | AGTCTGGCAGATAGCCAGGGTAAAACCGTTTATCATAGTCCGGG |
| | TGCACCGGATATTCGTGAATTTACCCGTGATGCAATTCCGGATA |
| | AAGATGCACAAGGTGGTGAAGTTTATCTGCTGAGCGGTCCGACC |
| | ATGATGATGCCTGGTCATGGTCATGGCCACATGGAACATAGCAA |
| | TTGGCGTATGATTAATCTGCCGGTTGGTCCGCTGGTTGATGGTA |
| | AACCGATTTATACCCTGTATATTGCCCTGAGCATTGATTTTCATC |
| | TGCACTATATCAACGATCTGATGAACAAACTGATTATGACCGCA |
| | AGCGTTATTAGCATCCTGATTGTTTTTATTGTTCTGCTGGCAGTT |
| | CATAAAGGTCATGCACCGATTCGTAGCGTTAGCCGTCAGATTCA |
| | GAATATTACCAGCAAAGATCTGGATGTTCGTCTGGATCCGCAGA |
| | CCGTTCCGATTGAACTGGAACAGCTGGTTCTGAGCTTTAATCAT |
| | ATGAGCGCAGAACTGGCAGAAAGCTATGCAGTTCTGGAACAGC |
| | GTGTTCAAGAGAAAACCGCAGGCCTGGAACATAAAAATCAGAT |
| | TCTGAGCTTTCTGTGGCAGGCAAATCGTCGTCTGCATAGCCGTG |
| | CACCGCTGTGTGAACGTCTGAGTCCGGTTCTGAATGGTCTGCAG |
| | AACCTGACACTGCTGCGTGATATTGAACTGCGTGTTTATGATAC |
| | CGATGATGAAGAAAACCACCAAGAATTTACCTGTCAGCCGGAT |
| | ATGACCTGTGATGATAAAGGTTGTCAGCTGTGTCCGCGTGGTGT |
| | TCTGCCGGTGGGTGATCGTGGTACAACCCTGAAATGGCGTCTGG |
| | CCGATAGTCATACCCAGTATGGTATCCTGCTGGCGACACTGCCG |
| | CAGGGTCGTCATCTGAGCCATGATCAGCAGCAGCTGGTAGATAC |
| | CCTGGCCGAACAGCTGACCGCAACACTGGCACTGGATCGTCATC |
| | AAGAACGTCAGCAGCAACTGATTGTTATGGAAGAACGTGCCAC |
| | CATTGCGCGTGAACTGCATGATAGCATTGCACAGAGCCTGAGCT |
| | GTATGAAAATGCAGGTTAGCTGTCTGCAGATGCAGGGTGATGCA |
| | CTGCCGGAAAGCTCACGCGAACTGCTGAGCCAGATTCGCAATG |
| | AACTGAATGCCAGCTGGGCACAGCTGCGTGAGCTGCTGATTACC |
| | TTTCGTCTGCAGCTGACAGAACCGGGTCTGCGTCCGGCACTGGA |
| | AGCAAGCTGTGAAGAATATTCAGCGAAATTTGGTTTTCCGGTGA |
| | AACTGGATTATCAGCTGCCTCCGCGTCTGGTTCCGAGCCATCAG |
| | GCAATTCATCTGCTGCAAATTGCACGTGAAGCACTGAGCAATGC |
| | ACTGAAACATTCACAGGCAAGCGAAGTTGTTGTTACCGTTGCAC |
| | AGAATGATAACCAGGTTAAACTGACCGTTCAGGATAATGGTTGT |
| | GGTGTTCCGGAAAATGCAATTCGTAGCAATCATTATGGCATGAT |
| | CATTATGCGTGATCGTGCCCAGAGCCTGCGTGGTGATTGTCGTG |
| | TTCGTCGTCGTGAAAGCGGTGGTACAGAAGTGGTGGTTACCTTT |
| | ATTCCTGAGAAAACCTTTACCGATGTTCAGGGTGATACCCATGA |
| | AGGTGGTGGTAGCGAACAGAAACTGATTTCAGAAGAAGATCTG |
| | TAA |
| NarX-NrsS | ATGAATACCCGTCGTCTGTTTGCACGTAGCCGTCTGCAGCTGGC |
| Chimera | ATTTTGGTATGCACTGGTTATGGGTGGTATTCTGACCCTGTTAGG |
| (SEQ ID NO: | TCTGGGTGTTTATCGTGCAATTGTTCAGGCAAATTGGATGGCAC |
| 6) | TGGAACGTGAAGTTGAAAGCATTGCAGGCACCCTGCATGATAG |
| | CCTGGAACCGATGCTGCCGAGCAATGCAAGCCCGACCGGTGTG |
| | CTGCAGAAAATGCTGCCGGATCTGTGTCTGGTTAATCAGCCGTG |
| | CCAGGTTAATCCGACACTGATTGAACGTCATACCCTGGGTATTA |
| | GCGATCGTAGCCTGTATTATATCCGCCTGTTTGATTATCAGGGTA |
| | ATCTGCTGGCGTTTAGCCCGAATCAGCCTGCAAGCCTGAGCAGC |
| | ATCTTTAATCAAGAAACCTGGCAGACCATTCATCCGCCTACCGG |
| | TGATCGTTATCGTCAGTTTACCACCATTCTGCATAGCGCAGGTA |
| | ATACCGATAAAAGCAGCTGGGGTTATCTGCAAATTGGTCGTAGT |
| | CTGGCAGCCTTTGATGCCGAAAATAAACGTATTCTGTGGATTCT |
| | GGGTCTGAGCTTTCCGATTGCACTGGGTTTAGTTGCATTTAGCA |
| | GTTGGGGTTTAGCAGGTCTGGCAATGCGTCCGATTTATCAGAGC |
| | TATCAGCAGCAGCAACAGTTTACCGCAAATGCAGCACATGAACT |
| | GCGTAGTCCGCTGGCAAGCCTGCTGGCAACCGTTGAAGCAGTTC |
| | TGCGTATTGATAGCAGCCATAGTCCGGAAATTAACACCATGTCT |
| | GCAGAACTGGCCGAAAGTTATGCCGTACTTGAGCAGCGGGTTCA |
| | GGAGAAAACCGCCGGGCTGGAGCATAAAAATCAGATCCTCTCT |
| | TTTTTATGGCAGGCTAACCGCCGTTTGCATTCCCGCGCCCCGCTG |
| | TGTGAACGCCTGTCACCTGTACTCAACGGCTTACAGAATTTAAC |
| | CCTGCTACGTGATATCGAATTGCGGGTGTATGACACTGATGATG |
| | AAGAGAATCATCAGGAGTTTACCTGCCAGCCAGATATGACTTGT |
| | GATGATAAAGGCTGCCAGCTCTGCCCGCGCGGCGTATTACCCGT |
| | TGGTGATCGCGGCACGACCCTGAAGTGGCGGCTGGCTGACTCTC |
| | ATACGCAGTACGGTATTTTGCTGGCGACCCTGCCACAGGGGCGT |
| | CATCTTAGCCATGATCAACAACAACTGGTGGATACCCTGGCTGA |
| | ACAACTCACCGCCACGCTGGCGCTGGATCGCCATCAGGAACGTC |
| | AGCAACAGTTGATCGTGATGGAAGAGCGTGCCACCATTGCGCG |
| | CGAACTGCATGATTCTATTGCCCAATCTCTCTCTTGCATGAAGAT |
| | GCAGGTGAGTTGTTTACAGATGCAGGGCGATGCGCTGCCAGAA |
| | AGCAGCCGCGAACTGTTAAGTCAGATCCGTAACGAACTGAATG |
| | CATCCTGGGCGCAGTTGCGTGAATTGCTCATCACATTCCGCTTG |
| | CAGCTCACCGAGCCTGGATTACGTCCGGCGCTGGAGGCGAGTTG |
| | CGAAGAGTACAGCGCCAAATTTGGCTTCCCGGTGAAGCTGGATT |
| | ATCAATTGCCGCCTCGCCTGGTGCCTTCGCATCAGGCAATCCAC |
| | TTGTTGCAAATTGCCCGTGAGGCATTAAGTAACGCCCTCAAACA |
| | TTCGCAAGCGAGTGAAGTCGTGGTGACGGTGGCGCAAAACGAT |
| | AATCAGGTCAAACTGACCGTCCAGGATAACGGCTGCGGCGTGC |
| | CTGAAAATGCCATCCGCAGCAATCACTACGGCATGATAATAATG |
| | CGCGATCGTGCGCAAAGTTTACGAGGCGATTGCCGCGTCCGCCG |
| | TCGTGAATCAGGTGGCACCGAAGTGGTGGTCACCTTTATTCCCG |
| | AAAAAACTTTCACAGACGTCCAAGGAGATACCCATGAGGGAGG |
| | AGGAAGCGAGCAGAAACTCATCTCTGAAGAGGATCTGTAA |
| NarX-VanS | ATGGTGATCAAGCTGAAGAACAAGAAAAACGACTACAGCAAAC |
| Chimera | TGGAACGCAAGCTGTATATGTATATTGTTGCCATTGTTGTGGTG |
| (SEQ ID NO: | GCCATTGTGTTTGTTCTGTATATTCGTAGCATGATCCGTGGCAAA |
| 7) | TTAGGTGATTGGATTCTGAGCATTCTGGAAAACAAATATGATCT |
| | GAATCACCTGGATGCCATGAAACTGTATCAGTATAGCATTCGCA |
| | ACAACATCGACATCTTTATCTATGTGGCGATTGTGATTAGCATTC |
| | TGATTCTGTGTCGTGTGATGCTGAGTAAATTCGCCAAATATTTCG |
| | ATGAAATCAACACCGGCATTGATGTGCTGATTCAGAATGAAGAT |
| | AAGCAGATTGAACTGAGCGCAGAAATGGATGTTATGGAACAGA |
| | AACTGAACACCATGTCTGCAGAACTGGCCGAAAGTTATGCCGTA |
| | CTTGAGCAGCGGGTTCAGGAGAAAACCGCCGGGCTGGAGCATA |
| | AAAATCAGATCCTCTCTTTTTTATGGCAGGCTAACCGCCGTTTGC |
| | ATTCCCGCGCCCCGCTGTGTGAACGCCTGTCACCTGTACTCAAC |
| | GGCTTACAGAATTTAACCCTGCTACGTGATATCGAATTGCGGGT |
| | GTATGACACTGATGATGAAGAGAATCATCAGGAGTTTACCTGCC |
| | AGCCAGATATGACTTGTGATGATAAAGGCTGCCAGCTCTGCCCG |
| | CGCGGCGTATTACCCGTTGGTGATCGCGGCACGACCCTGAAGTG |
| | GCGGCTGGCTGACTCTCATACGCAGTACGGTATTTTGCTGGCGA |
| | CCCTGCCACAGGGGCGTCATCTTAGCCATGATCAACAACAACTG |
| | GTGGATACCCTGGCTGAACAACTCACCGCCACGCTGGCGCTGGA |
| | TCGCCATCAGGAACGTCAGCAACAGTTGATCGTGATGGAAGAG |
| | CGTGCCACCATTGCGCGCGAACTGCATGATTCTATTGCCCAATC |
| | TCTCTCTTGCATGAAGATGCAGGTGAGTTGTTTACAGATGCAGG |
| | GCGATGCGCTGCCAGAAAGCAGCCGCGAACTGTTAAGTCAGAT |
| | CCGTAACGAACTGAATGCATCCTGGGCGCAGTTGCGTGAATTGC |
| | TCATCACATTCCGCTTGCAGCTCACCGAGCCTGGATTACGTCCG |
| | GCGCTGGAGGCGAGTTGCGAAGAGTACAGCGCCAAATTTGGCT |
| | TCCCGGTGAAGCTGGATTATCAATTGCCGCCTCGCCTGGTGCCT |
| | TCGCATCAGGCAATCCACTTGTTGCAAATTGCCCGTGAGGCATT |
| | AAGTAACGCCCTCAAACATTCGCAAGCGAGTGAAGTCGTGGTG |
| | ACGGTGGCGCAAAACGATAATCAGGTCAAACTGACCGTCCAGG |
| | ATAACGGCTGCGGCGTGCCTGAAAATGCCATCCGCAGCAATCAC |
| | TACGGCATGATAATAATGCGCGATCGTGCGCAAAGTTTACGAGG |
| | CGATTGCCGCGTCCGCCGTCGTGAATCAGGTGGCACCGAAGTGG |
| | TGGTCACCTTTATTCCCGAAAAAACTTTCACAGACGTCCAAGGA |
| | GATACCCATGAGGGAGGAGGAAGCGAGCAGAAACTCATCTCTG |
| | AAGAGGATCTGTAA |
| NarX-RssA | ATGATCGGCTTCAAAAGCTTCTTTATGCGCACCATTATCTTTCAG |
| (SEQ ID NO: | GTTCTGGCAATTCTGCTGCTGTGGGGTCTGCTGGTTGCATGGGT |
| 8) | GAAATATTGGTATTATCCGGACATGGAAAAATACTTTGATAACC |
| | AGCAGCGTATTGTTGCAGCAGGTATTGCAAATATTCTGGATGAA |
| | ACCGGCACCGACAATATTGATTATCGCGGTATTATCAAAACCAT |
| | CGAGGGCATGTATATCGACAGCATTAATAACGGTATGCAGGAT |
| | GAGATCGATTATCATCCGCTGTTTGTTGTGTATGATCGTGATAAT |
| | CGTGTGCTGTATAGCAGTCAGACCCAGGGTGAACCGCTGCGTCT |
| | GCCTCCGAGCGTTCTGAGCGGTAGCGTTAATTATGCCGGTGCAA |
| | ATTGGCATCTGGCAGGTAGCTGGAAAGAAAAACGTCAGTATCG |
| | TGTTATTGTGGGCGAAAGCTTTAATGATCGTACCACACTGTTTG |
| | GTAATCCGGCAGATGTTCCGCTGCTGGGTATTCTGGCAGCAATT |
| | ATTGTTACCCTGCTGTTTACCGCATATTTCAGCCTGCGTCCGCTG |
| | CGCCAGATTGCACGTACCATTAGCGATCGTCAGCCTGGTAATCT |
| | GAGCCCGATTAATGTTAGCGAACAGTATCAAGAAATTCGTCCGG |
| | TTGTTATGGAAGTGAACAAAATGTCTGCAGAACTGGCCGAAAGT |
| | TATGCCGTACTTGAGCAGCGGGTTCAGGAGAAAACCGCCGGGC |
| | TGGAGCATAAAAATCAGATCCTCTCTTTTTTATGGCAGGCTAAC |
| | CGCCGTTTGCATTCCCGCGCCCCGCTGTGTGAACGCCTGTCACCT |
| | GTACTCAACGGCTTACAGAATTTAACCCTGCTACGTGATATCGA |
| | ATTGCGGGTGTATGACACTGATGATGAAGAGAATCATCAGGAG |
| | TTTACCTGCCAGCCAGATATGACTTGTGATGATAAAGGCTGCCA |
| | GCTCTGCCCGCGCGGCGTATTACCCGTTGGTGATCGCGGCACGA |
| | CCCTGAAGTGGCGGCTGGCTGACTCTCATACGCAGTACGGTATT |
| | TTGCTGGCGACCCTGCCACAGGGGCGTCATCTTAGCCATGATCA |
| | ACAACAACTGGTGGATACCCTGGCTGAACAACTCACCGCCACGC |
| | TGGCGCTGGATCGCCATCAGGAACGTCAGCAACAGTTGATCGTG |
| | ATGGAAGAGCGTGCCACCATTGCGCGCGAACTGCATGATTCTAT |
| | TGCCCAATCTCTCTCTTGCATGAAGATGCAGGTGAGTTGTTTAC |
| | AGATGCAGGGCGATGCGCTGCCAGAAAGCAGCCGCGAACTGTT |
| | AAGTCAGATCCGTAACGAACTGAATGCATCCTGGGCGCAGTTGC |
| | GTGAATTGCTCATCACATTCCGCTTGCAGCTCACCGAGCCTGGA |
| | TTACGTCCGGCGCTGGAGGCGAGTTGCGAAGAGTACAGCGCCA |
| | AATTTGGCTTCCCGGTGAAGCTGGATTATCAATTGCCGCCTCGC |
| | CTGGTGCCTTCGCATCAGGCAATCCACTTGTTGCAAATTGCCCG |
| | TGAGGCATTAAGTAACGCCCTCAAACATTCGCAAGCGAGTGAA |
| | GTCGTGGTGACGGTGGCGCAAAACGATAATCAGGTCAAACTGA |
| | CCGTCCAGGATAACGGCTGCGGCGTGCCTGAAAATGCCATCCGC |
| | AGCAATCACTACGGCATGATAATAATGCGCGATCGTGCGCAAA |
| | GTTTACGAGGCGATTGCCGCGTCCGCCGTCGTGAATCAGGTGGC |
| | ACCGAAGTGGTGGTCACCTTTATTCCCGAAAAAACTTTCACAGA |
| | CGTCCAAGGAGATACCCATGAGGGAGGAGGAAGCGAGCAGAA |
| | ACTCATCTCTGAAGAGGATCTGTAA |

### Vesicle Preparation

Vesicles were prepared using the thin film hydration method. Briefly, lipid was deposited into a glass vial and dried with a stream of nitrogen and placed under vacuum for 3 hours. Films were then rehydrated in Milli-Q water for a minimum of 3 hours, and up to overnight. Vesicles were then vortexed and extruded 21× through a 100 nm polycarbonate filter.

### Cell-Free Protein Synthesis Reactions

Protein expression was performed using the myTxTl system (Arbor Biosceinces). 5 μL reactions were assembled with 10 mM lipid, 0.1 nM plasmid encoding T7 RNA polymerase, 9.9 nM plasmids encoding NarX, NarL, and reporter gene (nano luciferase or GFP) in specified ratios, and various concentration of ligands. Cell-free reactions were allowed to progress at 30° C. for 16 hours. Luciferase luminescence was read using the NanoGlo luciferase system and GFP fluorescence (ex. 480 nm, em. 507 nm) was read using a Molecular Devices Spectra Max i3 plate reader. To calculate fold change, luminescence values in the presence of nitrate were divided by the luminescence values in the absence of nitrate for each replicate.

### Western Blotting

1 μL of cell-free reaction was diluted to 15 μL in Laemlli buffer and heated at 95° C. for 10 minutes. Samples were then loaded and run on a 12% Mini-PROTEAN TGX Precast Protein Gel (Bio-Rad) at 150 V for 90 minutes. Wet transfer was performed onto a PVDF membrane (Bio-Rad) for 45 min at 100 V. Membranes were then blocked for an hour at room temperature in 5% milk in TBST (pH 7.6:50 mM Tris, 150 mM NaCl, HCl to pH 7.6, 0.1% Tween) and incubated for 1 hour at room temperature or overnight at 4° C. with primary solution (anti-Flag (Sigma F1804) or anti-Myc (ab32), diluted 1:1000 in 5% milk in TBST). Primary antibody solution was decanted, and the membrane was washed three times for 5 minutes in TBST and then incubated in secondary solution at room temperature for 1 hour (HRP-anti-Mouse (CST 7076) diluted 1:3000 in 5% milk in TBST). Membranes were then washed in TBST and incubated with Clarity Western ECL Substrate (Bio-Rad) for 5 min. Blots were imaged using an Azure Biosystems c280 imager and band intensities were quantified with ImageJ.

### Lyophilization of Reactions

Cell-free reactions were prepared as above, omitting vesicles and ligand. Reactions and vesicles were then flash frozen and lyophilized overnight. Following lyophilization, vesicles were rehydrated in water and cell free reactions were rehydrated in various media, such as Mili-Q water, Lake Water from Lake Michigan, and 50% FBS containing either 100 μM nitrate or an equivalent volume of water. Vesicles and reactions were allowed to rehydrate for 30 minutes on ice. Finally, vesicles were added to each reaction to bring the total volume to 5 μL and reactions were allowed to proceed at 30° C. for 16 hours.

Encapsulation of TCS into Vesicles

Cell-free machinery was encapsulated into vesicles using the water-in-oil emulsion method. A lipid film containing a molar ratio 1 POPC: 2 Cholesterol and 0.1 mol % rhodamine conjugate lipid was prepared. Films were then rehydrated with mineral oil and vortexed until the lipid was resuspended. The aqueous inner phase containing cell-free components and plasmids encoding NarX, NarL, and nanoluciferase or mEGFP were added to the lipid-oil mixture and briefly vortexed to produce an emulsion. Emulsions were then incubated for 5 minutes on ice and layered on top of outer solution (100 mM HEPES, 200 mM Glucose). The layered solutions were incubated on ice for 5 minutes and then centrifuged at 18,000 RCF for 15 minutes at 4° C. The top oil phase was removed with a pipette and the vesicle pellet was collected and transferred to a new tube containing an equal volume of outer solution. Vesicles were then centrifuged at 12,000 RCF for 5 minutes at 4° C. The vesicle pellet was then collected and divided into two. Outer solution containing RNAse and 1 mM nitrate was then added to 25 μL and samples were incubated at 30° C. for 16 hours.

For samples containing the luciferase plasmid, 0.5 μL of NanoGlo Luciferase substrate was added to the reaction, and reactions were read on a Molecular Devices Spectra Max i3 plate reader. For reactions containing a downstream mEGFP, 1 μL of the cell-free reaction was added to 100 μL of outer solution in a glass bottom 96 well plate (Corning). Samples were then imaged using a 20× objective on a Nikon confocal microscope. Images were analyzed using the Nikon NIS software.

### REFERENCES

1. A. D. Silverman, A. S. Karim, M. C. Jewett, Cell-free gene expression: an expanded repertoire of applications. Nature Reviews Genetics 2019 21:3 21, 151-170 (2019).
2. M. W. NIRENBERG, J. H. MATTHAEI, The dependence of cell-free protein synthesis in E. coli upon naturally occurring or synthetic polyribonucleotides. Proc Natl Acad Sci USA 47, 1588-1602 (1961).
3. A. C. Hunt, et al., A high-throughput, automated, cell-free expression and screening platform for antibody discovery. bioRxiv, 2021.11.04.467378 (2021).
4. K. F. Warfel, et al., A low-cost, thermostable, cell-free protein synthesis platform for on demand production of conjugate vaccines. bioRxiv, 2022.08.10.503507 (2022).
5. J. C. Stark, et al., On-demand biomanufacturing of protective conjugate vaccines. Sci Adv 7 (2021).
6. W. Thavarajah, et al., Point-of-Use Detection of Environmental Fluoride via a Cell-Free Riboswitch-Based Biosensor. ACS Synth Biol 9, 10-18 (2020).
7. J. K. Jung, et al., Cell-free biosensors for rapid detection of water contaminants. Nature Biotechnology 2020 38:12 38, 1451-1459 (2020).
8. P. Q. Nguyen, et al., Wearable materials with embedded synthetic biology sensors for biomolecule detection. Nature Biotechnology 2021 39:11 39, 1366-1374 (2021).
9. N. S. Kruyer, et al., Membrane Augmented Cell-Free Systems: A New Frontier in Biotechnology. ACS Synth Biol 10, 670-681 (2021).
10. V. Noireaux, A. Libchaber, A vesicle bioreactor as a step toward an artificial cell assembly. Proc Natl Acad Sci USA 101, 17669-17674 (2004).
11. K. P. Adamala, D. A. Martin-Alarcon, K. R. Guthrie-Honea, E. S. Boyden, Engineering genetic circuit interactions within and between synthetic minimal cells. Nature Chemistry 2016 9:59, 431-439 (2016).
12. M. A. Boyd, W. Thavarajah, J. B. Lucks, N. P. Kamat, Robust and tunable performance of a cell-free biosensor encapsulated in lipid vesicles. bioRxiv, 2022.03.02.482665 (2022).
13. J. M. Heili, et al., Controlled exchange of protein and nucleic acid signals from and between synthetic minimal cells. bioRxiv, 2022.01.03.474826 (2022).
14. J. A. Schoborg, et al., A cell-free platform for rapid synthesis and testing of active oligosaccharyltransferases. Biotechnol Bioeng 115, 739-750 (2018).
15. D. Blanken, D. Foschepoth, A. C. Serrão, C. Danelon, Genetically controlled membrane synthesis in liposomes. Nature Communications 2020 11:1 11, 1-13 (2020).
16. M. L. Jacobs, M. A. Boyd, N. P. Kamat, Diblock copolymers enhance folding of a mechanosensitive membrane protein during cell-free expression. Proc Natl Acad Sci USA 116, 4031-4036 (2019).
17. D. Toparlak, et al., Artificial cells drive neural differentiation. Sci Adv 6, 4920-4938 (2020).
18. I. Gispert, et al., Stimuli-responsive vesicles as distributed artificial organelles for bacterial activation. Proceedings of the National Academy of Sciences 119, e2206563119 (2022).
19. J. W. Hindley, et al., Building a synthetic mechanosensitive signaling pathway in compartmentalized artificial cells. Proc Natl Acad Sci USA 116, 16711-16716 (2019).
20. F. Jacob-Dubuisson, A. Mechaly, J. M. Betton, R. Antoine, Structural insights into the signalling mechanisms of two-component systems. Nature Reviews Microbiology 2018 16:10 16, 585-593 (2018).
21. J. T. Lazar, J. J. Tabor, Bacterial two-component systems as sensors for synthetic biology applications. Curr Opin Syst Biol 28, 100398 (2021).
22. P. Zhang, J. Yang, E. Cho, Y. Lu, Bringing Light into Cell-Free Expression. ACS Synth Biol 9, 2144-2153 (2020).
23. J. Yuan, F. Jin, T. Glatter, V. Sourjik, Osmosensing by the bacterial PhoQ/PhoP two-component system. Proc Natl Acad Sci USA 114, E10792-E10798 (2017).
24. B. P. Landry, R. Palanki, N. Dyulgyarov, L. A. Hartsough, J. J. Tabor, Phosphatase activity tunes two-component system sensor detection threshold. Nature Communications 2018 9:19, 1-10 (2018).
25. H. Liang, et al., Engineering bacterial two-component system PmrA/PmrB to sense lanthanide ions. J Am Chem Soc 135, 2037-2039 (2013).
26. S. R. Schmidl, et al., Rewiring bacterial two-component systems by modular DNA-binding domain swapping. Nature Chemical Biology 2019 15:7 15, 690-698 (2019).
27. W. R. Whitaker, S. A. Davis, A. P. Arkin, J. E. Dueber, Engineering robust control of two-component system phosphotransfer using modular scaffolds. Proc Natl Acad Sci USA 109, 18090-18095 (2012).
28. A. Mazé, Y. Benenson, Artificial signaling in mammalian cells enabled by prokaryotic two-component system. Nature Chemical Biology 2019 16:2 16, 179-187 (2019).
29. J. Hansen, et al., Transplantation of prokaryotic two-component signaling pathways into mammalian cells. Proc Natl Acad Sci USA 111, 15705-15710 (2014).
30. Estimated Nitrate Concentrations in Groundwater Used for Drinking|US EPA (Oct. 9, 2022).
31. R. Marshall, V. Noireaux, Synthetic biology with an all E. coli TXTL system: Quantitative characterization of regulatory elements and gene circuits. Methods in Molecular Biology 1772, 61-93 (2018).
32. M. Serizawa, J. Sekiguchi, The Bacillus subtilis YdfHI two-component system regulates the transcription of ydfJ, a member of the RND superfamily. Microbiology (NY) 151, 1769-1778 (2005).
33. J. M. Hershewe, et al., Improving cell-free glycoprotein synthesis by characterizing and enriching native membrane vesicles. Nature Communications 2021 12:1 12, 1-12 (2021).
34. J. J. Wuu, J. R. Swartz, High yield cell-free production of integral membrane proteins without refolding or detergents. Biochimica et Biophysica Acta (BBA)-Biomembranes 1778, 1237-1250 (2008).
35. M. C. Jewett, K. A. Calhoun, A. Voloshin, J. J. Wuu, J. R. Swartz, An integrated cell-free metabolic platform for protein production and synthetic biology. Mol Syst Biol 4, 220 (2008).
36. A. D. Silverman, N. Kelley-Loughnane, J. B. Lucks, M. C. Jewett, Deconstructing Cell-Free Extract Preparation for in Vitro Activation of Transcriptional Genetic Circuitry. ACS Synth Biol 8, 403-414 (2019).
37. C. E. Hilburger, M. L. Jacobs, K. R. Lewis, J. A. Peruzzi, N. P. Kamat, Controlling Secretion in Artificial Cells with a Membrane and Gate. ACS Synth Biol 8, 1224-1230 (2019).
38. M. L. Jacobs, et al., Probing the force-from-lipid mechanism with synthetic polymers. bioRxiv, 2022.05.20.492859 (2022).
39. M. G. Gutierrez, K. S. Mansfield, N. Malmstadt, The Functional Activity of the Human Serotonin 5-HT1A Receptor Is Controlled by Lipid Bilayer Composition. Biophys J 110, 2486-2495 (2016).
40. S. Ballweg, et al., Regulation of lipid saturation without sensing membrane fluidity. Nature Communications 2020 11:1 11, 1-13 (2020).
41. J. A. Peruzzi, et al., Hydrophobic mismatch drives self-organization of designer proteins into synthetic membranes. bioRxiv, 2022.06.01.494374 (2022).
42. N. J. Harris, et al., Structure formation during translocon-unassisted co-translational membrane protein folding. Scientific Reports 2017 7:1 7, 1-15 (2017).
43. M. R. Sanders, H. E. Findlay, P. J. Booth, Lipid bilayer composition modulates the unfolding free energy of a knotted α-helical membrane protein. Proc Natl Acad Sci USA 115, E1709-E1808 (2018).
44. J. Steinküher, et al., Improving cell-free expression of membrane proteins by tuning ribosome co-translational membrane association and nascent chain aggregation. bioRxiv, 2023.02.10.527944 (2023).
45. B. Mensa, et al., Allosteric mechanism of signal transduction in the two-component system histidine kinase PhoQ. Elife 10 (2021).
46. K. Pluhackova, A. Horner, Native-like membrane models of E. coli polar lipid extract shed light on the importance of lipid composition complexity. BMC Biology 2021 19:1 19, 1-22 (2021).
47. R. Utsumi, et al., Activation of Bacterial Porin Gene Expression by a Chimeric Signal Transducer in Response to Aspartate. Science (1979) 245, 1246-1249 (1989).
48. K. A. Kowallis, S. W. Duvall, W. Zhao, W. S. Childers, “Manipulation of Bacterial Signaling Using Engineered Histidine Kinases” in (2020), pp. 141-163.
49. J. M. Skerker, et al., Rewiring the Specificity of Two-Component Signal Transduction Systems. Cell 133, 1043-1054 (2008).
50. G. P. Munson, D. L. Lam, F. W. Outten, T. v. O'Halloran, Identification of a Copper-Responsive Two-Component System on the Chromosome of Escherichia coli K-12. J Bacteriol 182, 5864-5871 (2000).
51. C.-S. Lin, et al., An iron detection system determines bacterial swarming initiation and biofilm formation. Sci Rep 6, 36747 (2016).
52. M. Arthur, C. Molinas, P. Courvalin, The VanS-VanR two-component regulatory system controls synthesis of depsipeptide peptidoglycan precursors in Enterococcus faecium BM4147. J Bacteriol 174, 2582-2591 (1992).
53. L. Lopez-Maury, M. Garcia-Dominguez, F. J. Florencio, J. C. Reyes, A two-component signal transduction system involved in nickel sensing in the cyanobacterium Synechocystis sp. PCC 6803. Mol Microbiol 43, 247-256 (2002).
54. N. Krinsky, et al., Synthetic Cells Synthesize Therapeutic Proteins inside Tumors. Adv Healthc Mater 7, 1701163 (2018).
55. M. E. Inda, et al., A lipid-mediated conformational switch modulates the thermosensing activity of DesK. Proc Natl Acad Sci USA 111, 3579-3584 (2014).
56. I. Levental, E. Lyman, Regulation of membrane protein structure and function by their lipid nano-environment. Nature Reviews Molecular Cell Biology 2022, 1-16 (2022).
57. J. T. Marinko, et al., Folding and Misfolding of Human Membrane Proteins in Health and Disease: From Single Molecules to Cellular Proteostasis. Chem Rev 119, 5537-5606 (2019).
58. S. Berhanu, T. Ueda, Y. Kuruma, Artificial photosynthetic cell producing energy for protein synthesis. Nature Communications 2019 10:1 10, 1-10 (2019).
59. H. C. Lai, et al., The RssAB two-component signal transduction system in Serratia marcescens regulates swarming motility and cell envelope architecture in response to exogenous saturated fatty acids. J Bacteriol 187, 3407-3414 (2005).
60. C. W. Koo, J. M. Hershewe, M. C. Jewett, A. C. Rosenzweig, Cell-Free Protein Synthesis of Particulate Methane Monooxygenase into Nanodiscs. ACS Synth Biol 11, 4009-4017 (2022).
61. A. D. Silverman, U. Akova, K. K. Alam, M. C. Jewett, J. B. Lucks, Design and Optimization of a Cell-Free Atrazine Biosensor. ACS Synth Biol 9, 671-677 (2020).
62. H. J. Jackson, S. Rafiq, R. J. Brentjens, Driving CAR T-cells forward. Nat Rev Clin Oncol 13, 370-383 (2016).
63. S. H. Park, A. Zarrinpar, W. A. Lim, Rewiring MAP kinase pathways using alternative scaffold assembly mechanisms. Science (1979) 299, 1061-1064 (2003).
64. J. Hansen, Y. Benenson, Synthetic biology of cell signaling. Nat Comput 15, 5-13 (2016).
65. Y. Benenson, Biomolecular computing systems: Principles, progress and potential. Nat Rev Genet 13, 455-468 (2012).
66. A. Mazé, Y. Benenson, Artificial signaling in mammalian cells enabled by prokaryotic two-component system. Nat Chem Biol 16, 179-187 (2020).
67. T. Strittmatter, et al., Programmable DARPin-based receptors for the detection of thrombotic markers. Nature Chemical Biology 2022, 1-10 (2022).

1. A cell-free system comprising a nucleic acid encoding a histidine kinase of a two-component system; a nucleic acid encoding a response regulator of the two-component system; and at least one nucleic acid encoding a target molecule operably linked to a response regulator-binding promoter.

2. The system of claim 1, wherein the nucleic acid encoding the histidine kinase is a NarX-encoding nucleic acid and the nucleic acid encoding the response regulator is a NarL-encoding nucleic acid.

3. The system of claim 2, wherein the NarX-encoding plasmid and the NarL-encoding plasmid are at a ratio of between 1:3 and 10:1.

4. The system of claim 2, wherein the NarX-encoding nucleic acid comprises a sequence having at least 90% identity to SEQ ID NO: 1, SEQ ID NO: 6, or SEQ ID NO: 8.

5. The system of claim 1, further comprising a synthetic membrane, wherein the synthetic membrane comprises at least one of DMPC, POPC, DOPC, POPE, POPG, and E. coli polar lipid extract.

6. The system of claim 5, wherein the synthetic membrane comprises 90% POPC and 10% of one of POPE and POPG.

7. A method of detecting an analyte in a sample, the method comprising:
adding to the sample: a synthetic membrane and a cell-free system, the cell-free system comprising:
a nucleic acid encoding a histidine kinase of a two-component system, wherein the histidine kinase binds the analyte;
a nucleic acid encoding a response regulator for the two-component system; and
at least one nucleic acid encoding a reporter molecule operably linked to a response regulator-binding promoter; and
detecting the reporter molecule.

8. The method of claim 7, wherein the nucleic acid encoding the histidine kinase is a NarX-encoding nucleic acid and the nucleic acid encoding the response regulator is a NarL-encoding nucleic acid.

9. The method of claim 8, wherein the analyte is nitrate and the NarX-encoding nucleic acid comprises a sequence having at least 90% identity to SEQ ID NO: 1.

10. The method of claim 8, wherein the analyte is nickel and the NarX-encoding nucleic acid comprises a sequence having at least 90% identity to SEQ ID NO: 6.

11. The method of claim 8, wherein the analyte is iron and the NarX-encoding nucleic acid comprises a sequence having at least 90% identity to SEQ ID NO: 8.

12. The method of claim 7, wherein the reporter molecule comprises luciferase or EGFP.

13. The method of claim 7, wherein the NarX-encoding nucleic acid; the NarL-encoding nucleic acid, and the nucleic acid encoding the reporter molecule are plasmids.

14. The method of claim 13, wherein the NarX-encoding plasmid and the NarL-encoding plasmid are at a ratio of between 1:3 and 10:1.

15. The method of claim 7, wherein the synthetic membrane comprises at least one of DMPC, POPC, DOPC, POPE, POPG, and E. coli polar lipid extract.

16. The method of claim 15, wherein the synthetic membrane comprises 90% POPC and 10% of one of POPE and POPG.

17. The method of claim 7, wherein the synthetic membrane comprises 1 POPC: 2 cholesterol; and wherein the synthetic membrane further comprises a rhodamine conjugate lipid.

18. The method of claim 7, further comprising incubating the sample, the synthetic membrane, and the cell-free system for at least about 6 hours at about 30° C. before detecting the reporter molecule.

19. A method of synthesizing a target molecule in a sample, the method comprising:
adding to the sample: a synthetic membrane, and a cell-free system, the cell-free system comprising:
a nucleic acid encoding a histidine kinase of a two-component system;
a nucleic acid encoding a response regulator of the two-component system; and
at least one nucleic acid encoding a target molecule operably linked to a response regulator-binding promoter; and
adding an activator to the sample, wherein the histidine kinase binds the activator.

20. A kit comprising the system of claim 5, wherein the synthetic membrane is separate from the NarX-encoding nucleic acid, the NarL-encoding nucleic acid, and the at least one nucleic acid encoding the target molecules.
